SEQUENCE LISTING (4 sequences)

SEQ ID NO: 1 - DNA, 20 bp, Mus musculus
  misc_feature 1..20: note = Sense primer
  source 1..20: mol_type = other DNA
gtgggtatgggctgcttttt

SEQ ID NO: 2 - DNA, 21 bp, Mus musculus
  misc_feature 1..21: note = Anti-sense primer
  source 1..21: mol_type = other DNA
cgtgtattcgcttggtgacat

SEQ ID NO: 3 - DNA, 20 bp, Mus musculus
  misc_feature 1..20: note = Sense primer
  source 1..20: mol_type = other DNA
gtaacccgttgaaccccatt

SEQ ID NO: 4 - DNA, 20 bp, Mus musculus
  misc_feature 1..20: note = Anti-sense primer
  source 1..20: mol_type = other DNA
ccatccaatcggtagtagcg

(19) United States
(12) Patent Application Publication
(10) Pub. No.: US 2023/0036161 A1
(43) Pub. Date: Feb. 2, 2023

(54) AGENT FOR USE IN THE TREATMENT AND PROPHYLAXIS OF POSTISCHEMIC TISSUE INJURY

(71) Applicants: Eberhard Karls Universität Tübingen Medizinische Fakultät, Tübingen (DE); Julius-Maximilians-Universität Würzburg, Würzburg (DE)

(72) Inventors: Peter Rosenberger, Tübingen (DE); Bernhard Nieswandt, Eibelstadt (DE)

(21) Appl. No.: 17/819,465

(22) Filed: Aug. 12, 2022

Related U.S. Application Data
(63) Continuation of application No. PCT/EP2021/053464, filed on Feb. 12, 2021.

(30) Foreign Application Priority Data
Feb. 14, 2020 (DE) 10 2020 103 987.5

(51) Int. Cl. C07K 16/28; A61P 9/10

(52) U.S. Cl. CPC C07K 16/2803; A61P 9/10; A61K 2039/505

(57) ABSTRACT

The present invention relates to an agent for use in the treatment and/or prophylaxis of postischemic tissue damage, a pharmaceutical composition containing said agent, a method for the preparation of a medicament for the treatment and/or prophylaxis of postischemic tissue damage, and a method for the treatment and/or prophylaxis of postischemic tissue damage.

### CROSS REFERENCE TO RELATED APPLICATIONS

This application is a continuation of PCT/EP2021/053464, filed Feb. 12, 2021, which claims the benefit of the priority of German Patent Application No. 102020103987.5, filed Feb. 14, 2020, the entire contents of each are incorporated herein by reference.

### INCORPORATION BY REFERENCE OF SEQUENCE LISTING

This application incorporates by reference the material contained in the Sequence Listing XML file being submitted concurrently herewith: File name: WWEL109.002C1_ST26.xml; created Aug. 11, 2022, 5,764 bytes in size.

### FIELD OF THE INVENTION

The present invention relates to an agent for use in the treatment and/or prophylaxis of postischemic tissue damage, a pharmaceutical composition containing said agent, a method for the preparation of a medicament for the treatment and/or prophylaxis of postischemic tissue damage, and a method for the treatment and/or prophylaxis of postischemic tissue damage.

The invention relates to the field of molecular medicine, and more particularly to the field of prophylaxis and therapy of post-ischemic tissue damage.

### BACKGROUND OF THE INVENTION

Myocardial infarction (MI) is still one of the most significant health problems worldwide. In the treatment of MI, early reperfusion of the myocardium is currently the most effective therapy to improve clinical outcome. However, reperfusion of previously ischemic myocardium may also induce tissue damage. This phenomenon, termed “reperfusion injury” (RI), reduces the beneficial effects of early reperfusion and is characterized by the infiltration of immune cells, mainly neutrophils, into previously ischemic areas where they contribute to damage through tissue inflammation. Platelets are increasingly recognized as central orchestrators of inflammatory processes, mainly by enhancing immune cell recruitment and modulating endothelial barrier function. This phenomenon is termed thrombo-inflammation. In the course of myocardial IR, the formation of complexes of platelets and neutrophils (platelet-neutrophil complexes; PNCs) worsens. The inflammatory tissue damage and is thus a marker for tissue inflammation.

Clinically, reperfusion injury leads to general tissue damage in the affected body segment and to hyperacidity or acidosis of the organism as a whole. Locally, this leads to hyperthermia, redness, and swelling of the affected section, e.g., leg or arm, up to the development of a compartment syndrome with extensive rhabdomyolysis. Generalized symptoms may range from mild acceleration of spontaneous breathing to hypotension, cardiac arrhythmias due to hyperkalemia, coagulation disorders, renal failure or even cardiovascular arrest.

### RELATED PRIOR ART

At present, the development of reperfusion damage can only be counteracted to a limited extent in a satisfactory manner. Thus, cooling down the affected tissue before reperfusion reduces the activity of the enzymes. Furthermore, the anesthesiologist directly counteracts metabolic acidosis during surgery by hyperventilation. In more severe cases, the acidosis is additionally buffered with sodium bicarbonate. In addition, circulatory supportive drugs such as catecholamines and diuretics are used if necessary. However, a targeted and specific treatment of postischemic tissue damage and especially reperfusion injury is currently not possible.

Against this background, the object underlying the invention is to develop a new prophylactic or therapeutic approach with which postischemic tissue damage can be counteracted or treated in a targeted manner. Preferably, a pharmaceutically active agent is to be provided with which the pathological mechanisms of post-ischemic tissue damage can be intervened in a regulatory manner.

### SUMMARY OF THE INVENTION

This object is solved by providing an inhibitor of semaphoring 7A (SEMA7A) for use in the treatment and/or prophylaxis of postischemic tissue injury.

Surprisingly, the inventors found that inhibition of SEMA7A can significantly counteract post-ischemic tissue damage, possibly even preventing it. This finding was surprising because SEMA7A had previously been described in a different context.

Semaphorin 7A (SEMA7A for the human variant, or Sema7a for the mouse variant, used synonymously here), GPI membrane anchor (John Milton Hagen blood group), also known as CD108 (Cluster of Differentiation 108), is a membrane-bound 4emaphoring associated with cell surfaces via glycosylphosphatidylinositol (GPI) binding [Entrez: 8482 (human); 20361 (mouse)]. SEMA7A is also known as the John Milton Hagen (JMH) blood group antigen, an 80 kD glycoprotein expressed on activated lymphocytes and erythrocytes. SEMA7A is known to be a receptor for the malaria parasite Plasmodium falciparum.

SEMA7A was originally described in the context of axonal growth as a messenger protein involved in the control of synapse formation for the neuronal circuit. Later work has shown that it enhances autoimmune encephalitis through T-cell-dependent cytokine production and can increase neutrophil infiltration in sites of tissue hypoxia. A role for SEMA7A in the cardiovascular system has recently been described in atherogenesis. In addition, it has been shown that impaired blood flow leads to induction of SEMA7A on the vascular endothelium and that this results in increased expression of leukocyte adhesion molecules on the endothelial surface.

The role of SEMA7A in platelet function is as yet unknown.

WO 2008/024300, WO 2009/133984, WO 2013/052631 and EP 3 220 447 propose inhibition of SEMA7A for the treatment of pulmonary fibrosis.

DE 20 2006 007 590 and US 2007/0264263 disclose that SEMA7A plays a role in the immune system. It is further disclosed that SEMA7A can cause activation of monocytes via its interaction with α1β1-integrin (VLA-1). It is proposed to use an antibody directed against VLA-1 to inhibit the interaction of VLA-1 with SEMA7A. This approach is suggested by the authors for the treatment of cytokine-mediated diseases, such as inflammatory diseases.

The inventors demonstrated in an experimental system that soluble SEMA7A is increased in the plasma of patients with acute myocardial infarction and that SEMA7A has a significant effect on the extent of postischemic tissue injury or reperfusion injury. They also demonstrated that SEMA7A promotes myocardial thrombo-inflammation and tissue injury by enhancing platelet thrombotic activity and PNC formation through a platelet GPlb-dependent mechanism. Conversely, the inventors notably found that inhibition of SEMA7A leads to reduced reperfusion injury.

According to the invention, “inhibition” is understood to mean any measure that leads to a reduction in the functionality of SEMA7A. This can be achieved, for example, by reducing the activity of SEMA7A, the amount and/or concentration of SEMA7A in the cellular membrane, transcription or translation, or other measures that result in a decrease in SEMA7A functionality. In one embodiment of the invention, the inhibition is directed directly against SEMA7A, but not against a binding partner of SEMA7A or the interaction of SEMA7A with such a binding partner, such as VLA-1.

According to the invention, “post-ischemic tissue damage” is generally understood to be a disease process that is triggered after a reduced blood flow or ischemia of a cellular tissue.

The problem underlying the invention is hereby completely solved.

In one embodiment of the invention, the postischemic tissue damage is reperfusion damage.

This measure has the advantage that one of the clinically particularly relevant reperfusion injuries can be effectively treated or prevented.

In another embodiment of the invention, the inhibitor is configured to modify the interaction of SEMA7A with the platelet surface, and preferably modifies the interaction of SEMA7A with GPlb.

This measure has the advantage that the invention directly interferes with the molecular mechanisms that the inventors have identified as playing a critical role in the development of postischemic tissue injury, such as myocardial reperfusion injury.

According to the invention, “GPlb” refers to glycoprotein lb, which is similar to a surface protein on platelets involved in blood clotting.

According to the invention, “modified” may mean “inhibited” in a first embodiment. In a further embodiment of the invention, “modified” may mean “activated” and/or “stimulated”.

In another embodiment of the invention, the inhibitor is selected from the group consisting of: antibody, antibody fragment, soluble SEMA7A receptor, antisense nucleic acid, siRNA, small molecule compound, and combinations thereof.

The measure has the advantage that such structures are used as inhibitors that are particularly suitable for the targeted inhibition of SEMA7A.

In a further embodiment of the invention, the α-SEMA7A antibody is a function-inhibiting antibody.

With this measure, such an antibody is used which selectively and specifically inhibits SEMA7A in its functionality and thus non-specific effects can be largely or completely avoided.

Another object of the invention relates to a pharmaceutical composition comprising the inhibitor according to the invention.

The features, advantages, further developments and embodiments of the inhibitor according to the invention apply equally to the pharmaceutical composition.

Pharmaceutically acceptable carriers are well known to those skilled in the art. They enable proper formulation of the inhibitor and serve to improve selectivity, efficacy and/or safety of drug delivery. Pharmaceutically acceptable carriers include, but are not limited to, solvents, fillers, binders, lubricants, stabilizers, surfactants, suspensions, thickeners, emulsifiers, preservatives, liposomes, micelles, microspheres, nanoparticles, etc., suitable for the particular dosage form. Materials that may serve as pharmaceutically acceptable carriers include, but are not limited to, monosaccharides and oligosaccharides and derivatives thereof; malt, gelatin; talc; excipients such as: cocoa butter and suppository waxes; oils such as peanut oil, cottonseed oil, safflower oil, sesame oil, olive oil, corn oil and soybean oil; glycols such as propylene glycol; esters such as ethyl oleate and ethyl laurate; agar; buffering agents such as magnesium hydroxide and aluminum hydroxide; alginic acid; pyrogen-free water; isotonic saline solution, Ringer's solution; ethyl alcohol and phosphate buffer solutions. In addition, the composition may contain other non-toxic compatible lubricants, for example, sodium lauryl sulfate and magnesium stearate, as well as colorants, release agents, film formers, sweeteners, flavor additives and flavorings, preservatives and antioxidants.

According to the invention, the pharmaceutical composition may comprise the SEMA7A inhibitor as the sole active ingredient, or may be present in combination with other active ingredients for the treatment and/or prophylaxis of post-ischemic tissue damage.

Another subject-matter of the present invention relates to the use of the inhibitor according to the invention for the treatment and/or prophylaxis of post-ischemic tissue damage.

The features, advantages, further developments and embodiments of the inhibitor according to the invention apply equally to the use according to the invention.

Another object of the invention relates to a method for the preparation of a medicament for the treatment and/or prophylaxis of post-ischemic tissue damage, comprising formulating an inhibitor of SEMA7A into a pharmaceutically acceptable carrier.

The features, advantages, further developments and embodiments of the inhibitor according to the invention apply equally to the manufacturing method according to the invention.

Another subject-matter of the present invention relates to a method for the treatment and/or prophylaxis of post-ischemic tissue injury comprising administering a therapeutically effective amount of an inhibitor of SEMA7A into a mammal in need, preferably a human, further preferably a myocardial infarction patient or a patient at risk of myocardial infarction.

The features, advantages, further developments and embodiments of the inhibitor according to the invention are equally applicable to the manufacturing process according to the invention.

In one embodiment of the method according to the invention, administration of the inhibitor modifies the interaction of SEMA7A with the platelet surface, and preferably, administration of the inhibitor modifies the interaction of SEMA7A with GPlb.

This measure has the advantage that the invention directly interferes with molecular mechanisms that the inventors have identified as playing a critical role in the development of post-ischemic tissue injury, such as myocardial reperfusion injury.

According to the invention, “modified” may mean “inhibited” in a first embodiment. In a further embodiment of the invention, “modified” may mean “activated” and/or “stimulated.”

Further advantages and features will be apparent from the following description of preferred embodiments and the accompanying drawings.

It is understood that the features mentioned above and to be explained below are usable not only in the respective combination indicated, but also in other combinations or on their own, without leaving the scope of the present invention.

### BRIEF DESCRIPTION OF THE FIGURES

FIG. 1: Patients with acute myocardial infarction show increased activation of PNCs and increased plasma SEMA7A concentration. Samples were collected from patients with acute myocardial infarction, patients undergoing cardiac surgery with the use of a heart-lung machine (HLM) or bypass surgery without the use of a heart-lung machine (off-pump), and healthy controls. A) Flow-cytometric evaluation of PNCs' expression of GPlb (CD42b) in the presented patient groups. B) Flow cytometric evaluation of PNCs for CD62P expression on neutrophils. I PAC-1 binding to PNCs. (d) Representative flow cytometric histograms of the measured groups, al(e) Sema7a in the plasma of patients with acute MI, patients undergoing cardiac surgical operations with (HLM) or without cardiopulmonary bypass (off-Pump) and healthy controls. For comparison, the inventors performed one-way analyses of variance followed by Dunnett's tests on the myocardial infarction group (data are means±SA; *p<0.05, **p<0.01, and *** p<0.001 as indicated; n≥4/group).

FIG. 2: Demographic and standard laboratory values of patients with myocardial infarction, cardiac surgery without cardiopulmonary bypass (off-pump), cardiopulmonary heart surgery (HLM) and healthy subjects.

FIG. 3: Expression of SEMA7A (%) on human erythrocytes in relation to age in years. Erythrocytes were analyzed at different ages to determine the level of SEMA7A expression on their surface. Comparison of SEMA7A (%) on human erythrocytes with donor years by ANOVA showed no significant differences in change over age, p=0.1194.

FIG. 4: Plasma levels of Sema7a in WT mice subjected to 1 minute of myocardial ischemia and reperfusion. WT mice were subjected to ischemia for 60 minutes. Samples were collected after 1 minute of reperfusion. Comparisons in FIG. 4 were analyzed by Student's t tests (data are means±SA; n≥3/group (*p<0.05 as indicated).

FIG. 5: SEMA7A cleavage from human erythrocytes. Human erythrocytes were subjected to either shear stress, hypoxia (2% O₂) or normoxia (21% O₂) for the indicated time points and SEMA7A concentrations were determined in the supernatant. For comparisons in FIG. 5, the inventors performed one-way analyses of variance followed by Dunnett's tests on the “5 min unstimulated” group (data are means±SA; n=4/group; *p<0.05, ***p<0.01 as indicated).

FIG. 6: Injection of Sema7a leads to increased myocardial RI injury and PNC formation. Mice were injected with either recombinant Semaphorin 7a (rmSema7a) or Fc control (rmlgG2A Fc) and then subjected to ischemia for 1 h followed by 2 h of reperfusion. Samples were collected after 1 min or 120 min of reperfusion. a) Representative TTC-stained sections of myocardial tissue with infarct area (blue/dark=retrograde Evans blue staining; red and white=AAR, white=infarct tissue; 120 min) with b) systematic evaluation of infarct sizes and corresponding troponin I plasma levels (120 I). (c) Representative histological sections of WT animals injected with either IgG Fc control or rmSema7a (120 min) and (d) number of PNCs counted in myocardial tissue sections of the AAR in animals (120 min). (e) Representative flow cytometric PNC plots in the blood of sham, rmSema7a, or IgG Fc control mice present GPlb (CD42b) and P-selectin (CD62P) after 1 min and 120 min of reperfusion. Systematic evaluation of flow cytometric expression of mean fluorescence intensity (MFI) for f) GPlb (CD42b) and g) P-selectin (CD62P) expression. h) Systematic evaluation of PNCs in % flow cytometrically in blood from animals injected with rmSema7a or Fc control after 1 min and 120 min. i) Representative flow cytometric plots of PNCs in the AAR showing the presence of GPlb (CD42b) and P-selectin (CD62P) after 1 min and 120 min of reperfusion. Systematic evaluation of flow cytometric expression of MFI for j) GPlb (CD42b) and k) P-selectin (CD62P) and I) systematic evaluation of PNCs in % flow cytometric in the AAR of animals injected with rmSema7a or Fc control at 1 min and 120 min. Comparisons in FIG. 6 b and d were analyzed by Student's t tests (data are mean±SD; n≥5/group; histology n=9/group). For FIG. 6 f, g, h, j, k, and l, the inventors used log-transformed data to match normality. For log-transformed data, tests were performed on the log values, and the results are shown as geometric mean values and their 95% confidence intervals (*p<0.05, **p<0.01, and *** p<0.001 as indicated).

FIG. 7: SEMA7A does not induce caspase 3 in human cardiac myocytes (HMCs). a) HMCs were exposed to recombinant human SEMA7A (rhSEMA7A), an appropriate Fc control (rhlgG1 Fc), BSA only, or staurosporine for the indicated times and caspase 3 expression was measured by ELISA. (b) HMCs were exposed to SEMA7A, the Fc control or BSA for 6 h and stained for caspase 3. Staurosporine treatment served as a positive control. Data are mean±SA; n≥4/group, histology n=3.

FIG. 8: Injection of Sema7a leads to enhanced αIIbβ3 activation of PNCs. Animals were injected with either rec12emaphoringemaphorin 7a (rmSema7a) or Fc control (rmlgG2A Fc) and then subjected to ischemia for 1 h followed by 2 h of reperfusion. Blood and myocardial tissue (AAR) sections were collected after 1 min or 120 min of reperfusion. Representative flow cytometric PNC plots in blood and tissue from sham, rmSema7a, or IgG Fc control injected mice showed αIIbβ3 (JON/A) activation. Systematic evaluation of flow cytometric mean fluorescence intensities (MFI) of αIIbβ3 (JON/A) on PNCs from rmSema7a-injected, IgG Fc control, or sham mice. For FIG. 8, the inventors used log-transformed data to fit normality. For log-transformed data, tests were performed on the log values, and the results are presented as geometric means and their 95% confidence intervals. (**p<0.01 and *** p<0.001 as indicated by n≥5).

FIG. 9: SEMA7A affects the migration of neutrophils in flow. Whole blood neutrophils treated with rhSEMA7A and labeled with rhodamine adhere to glass capillaries coated with E-selectin or E-selectin with ICAM1. a) Representative frame of neutrophil (magenta) traces (cyan) on coated capillaries with flow chamber experiments performed with whole samples from 4 independent healthy donors, and at least 150 cells were followed in each experimental group. b) Neutrophil velocity (μm/sec) on coated capillaries under the influence of rhSEMA7A. c) Neutrophils (magenta) and platelets (cyan) in flow on cremaster tissue before and 15 min after i.v. inoculation of rmSema7a were imaged. (d) From obtained intra-vital microscopy videos, neutrophil velocity (μm/sec) was calculated from the tracked cells; stationary neutrophils were counted (cell count/mm2); platelet sedimentation on the vessel wall was measured (normalized MFI %/mm); transmigrated cells from the vasculature after 15 min exposure to rmSema7a were counted (cell count/mm²); transmigrated cell spacing is measureln μm. (e) Representative optosplit image analysis of platelets (cyan) and neutrophils (magenta) moving on the mouse cremaster microvasculature (540 μm), with indications of how data are acquired from each 10-second image slot; white cross represents stationary cells; white lines connect (μm) traversed neutrophils to exit points from the vasculature; red lines describe the intensity profile of platelet attachment to the vasculature as mean fluorescence intensity (MFI); green circles track moving neutrophils over a defined region, which is translated into cell trajectory velocity (μm/sec.). Intavital microscopy experiments were performed on n≥4 mice with n≥10 videos per condition with 20-second intervals of rmSema7a groups recorded 15 minutes after baseline control. Data are presented as geometric mean with Cl, with P values denoted as *<0.05, **<0.01, ***<0.001; intravital microscopy experiments were performed on n=5 mice with videos of 10 seconds (n≥10 videos per condition with 20-second interval) from rmSema7a groups recorded 15 minutes after baseline control. Data are presented as geometric mean with Cl with P values labeled *<0.05, **<0.01, ***<0.001.

FIG. 10: Sema7a^{-/-}animals show reduced signs of myocardial RI injury and attenuated PNC formation. a) Sema7a^{-/-}animals and littermate controls were evaluated by magnetic resonance imaging (MRI) to exclude anatomical and functional changes in Sema7a^{-/-}animals. b) Left and right ventricular ejection fraction (LVEF or RVEF in %) were determil by MRI. (c) Sema7a^{-/-}mice and littermate controls were subjected to 60 minutes of ischemia and 120 minutes of reperfusion, with samples collected after 1 minute or 120 minutes of reperfusion. Representative TTC-stained sections of myocardial tissue showing infarct area (blue/dark=retrograde Evans blue staining; red and white=AAR, white=infarct tissue, 120 min) with (d) systematic evaluation of infarct sizes and corresponding troponin I plasma levl (120 min). (e) Representative histological sections of Sema7a^{-/-}and littermate control hearts (120 min). (f) Number of PNCs in myocardial tissue sections in the AAR of Sema7a^{-/-}mice and littermate controls (120 min). (g) Representative flow cytometric PNC plots in blood from Sema7a^{-/-}and littermate controls showing GPlb (CD42b) and P-selectin (CD62P) after 1 min and 120 min of reperfusion. Mean fluorescence intensity (MFI) for h) GPlb (CD42b) and i) P-selectin (CD62P) in PNCs and j) systematic evaluation of PNCs in % by flow cytometry in blood from Sema7a^{-/-}animals and littermate controls. k) Representative flow cytometric plots of PNCs in the AAR and MFI for I) GPlb (CD42b) and m) P-selectin (CD62P) and n) systematic evaluation of PNCs by flow cytometry in % in the AAR of WTs and Sema7a^{-/-}animals at 1 min and 120 min. Comparisons in FIG. 10 b, d, and f were analyzed by Student's t-tests (data are means±SA; n≥5/group; histology n=9/group). For FIG. 10 h, i, j, l, m, and n, the inventors used log transformation of the data to fit normality. For log-transformed data, tests were performed on the log values, and the results are shown as geometric means and their 95% confidence intervals (*p<0.05, **p<0.01, and *** p<0.001 as indicated).

FIG. 11: Sema7a^{-/-}animals show no altered cardiac performance compared to WT littermates. Short-axis cardiac MRI images were acquired on a 70/39 7T BioSpec scanner using the IntraGateFLASH measurement method with the following parameters: Echo time=2.112 ms, 100 repetitions of 74.352 ms, 128-fold square matrix, spatial resolution of 176 μm. Ten axial slices of 1 mm thickness and 10 cardiac frames were taken for reconstruction to cover RV and LV. a) Assessment of left ventricular end-diastolic volume (LVEDV), b) left ventricular end-systolic volume (LVESV), c) left ventricular mass (LV mass), and d) left ventricular stroke vole (LV stroke). (e) Assessment of right ventricular end-diastolic volume (RVEDV), (f) right ventricular end-systolic volume (RVESV), (g) right ventricular mass (RV mass), and (h) right ventricular stroke volume (RV stroke). Comparisons were analyzed by Student's t tests (data are means±SA; n=6/group (*p<0.05 as indicated).

FIG. 12: Sema7a^{-/-}animals showed reduced signs of αIIbβ3 (JON/A) activation in PNCs. Sema7a^{-/-}and littermate controls were subjected to ischemia for 1 h and to reperfusion for 2 h. Blood and myocardial tissue (AAR) samples were collected after 1 min or 120 min of reperfusion. Representative flow cytometric plots of PNCs in blood and tissue from sham animals, Sema7a−/−, and littermate controls showed activated αIIbβ3 (JON/A) activation in PNCs. Systematic evaluation of mean fluorescence intensities of αIIbβ3 (JON/A) on PNCs from Sema7a^{-/-}and littermate controls. For FIG. 12, the inventors used log-transformed data to match normality. For log-transformed data, tests were performed on the log values, and the results are shown as geometric means and their 95% confidence intervals. (*p<0.05 and **p<0.01 as indicated by n≥5/group).

FIG. 13: Sema7a expression in mouse tissues and human Hematopoietic cells. a) Relative Sema7a mRNA expression in mouse tissues compared to brain. b) Densitometry of Sema7a protein expression in mouse tissues with a representative Wlern blot imaging. (c) Densitometry of Sema7a protein expression on human hematopoietic cells with a representative western blot image. RBC=red blood cells, Mono=monocytes, Plt=platelets, and PMNs=polymorphonuclear cells. (All data are mean±SA; n≥4/group).

FIG. 14: RBC-derived semaphorin 7A drives PNC formation and worsens myocardial RI injury. Sema7a^{loxP/loxP} HBBCre+, Sema7a^{loxP/loxP} Myh6Cre+, Sema7a^{loxP/loxP} Tie2Cre+ and Sema7a^{loxP/loxP} LysMCre+ animals or littermate controls were subjected to ischemia for 60 minutes and reperfusion for 120 minutes. (a) Representative TTC-stained sections of myocardial tissue showing infarcted area (blue/dark=retrograde Evans blue staining; red and white=AAR, white=infarcted tissue) in Sema7a^{loxP/loxP} HBBCre+ or littermate controls with (b) systematic evaluation of infarct sizes and correlating troponin I plasma levels. (c) Representative histological sections from Sema7a^{loxP/loxP} HBBCre+ animals or littermate controls, and (d) number of PNCs counted from myocardial AAR sections in Sema7a^{loxP/loxP} HBBCre+ animals littermate controls. (e) Representative TTC-stained sections of myocardial tissue showing the infarct area in Sema7aloxP/loxPMyh6Cre+ or littermate controls with (f) systematic evaluation of infarct sizes and correlating troponin I plasma levels. g) Representative histological sections from Sema7a^{loxP/loxP} Myh6Cre+ animals or littermate controls. h) The number of PNCs counted in myocardial AAR sections in Sema7a^{loxP/loxP} Myh6Cre+ animals or littermate controls. i) Representative TTC-stained cardiac sections showing myocardial infarctions in Sema7a^{loxP/loxP} Tie2Cre+ or littermate controls with j) systematic evaluation of infarct sizes and correlating troponin I plasma levels. k) Representative histological sections from Sema7a^{loxP/loxP} Tie2Cre+ animals or littermate controls, and l) number of PNCs counted in myocardial tissue sections from Sema7a^{loxP/loxP} Tie2Cre+ or littermate controls. (m) Representative TTC-stained sections of myocardial tissue showing infarct area in Sema7a^{loxP/loxP} LysMCre+ or littermate controls, with (n) systematic evaluation of infarct sizes and correlating troponin I plasma levels. o) Representative histological sections from Sema7a^{loxP/loxP} LysMCre+ animals or littermate controls and p) number of PNCs counted from myocardial tissue sections from Sema7a^{loxP/loxP} LysMCre+ or littermate controls. All comparisons in FIG. 14 were analyzed by Student's t tests (data are means±SA; n≥5/group; histology n=9/group). (*p<0.05, **p<0.01, and *** p<0.001 as indicated).

FIG. 15: Reconstitution of Sema7a^{loxP/loxP} HBBCre+ results in increased MIRI. Sema7a^{loxP/loxP} HBBCre+ animals or littermate controls were inject16emaphoringther recombinant semaphorin 7a (rmSema7a) or Fc control (rmlgG2A Fc) and then subjected to ischemia for 1 h followed by 2 h of reperfusion. (a) Representative TTC-stained sections of myocardial tissue showing the infarcted area (blue/dark=retrograde Evans blue staining; red and white=AAR, white=infarcted tissue). (b) Systematic evaluation of infarct sizes and correlating troponin I plasma levels. (c) Representative histological sections stained for the presence of PNCs; and (d) number of PNCs counted from myocardial AAR sections in Sema7a^{loxP/loxP} HBBCre+ animals inject16emaphoringther recombinant semaphorin 7a (rmSema7a) or Fc control (rmlgG2A Fc). All comparisons in FIG. 15 were analyzed by Student's t tests (data are means±SA; n≥5/group; histology n=9/group). (*p<0.05 and *** p<0.001 as indicated).

FIG. 16: Sema7a exerts its function via platelet glycoprotein lb (GPlb). a) rmSema7a markedly enhances adhesion and thrombus formation of WT platelets on collagen in flow at a shear rate of 1000 s⁻¹. Blockade of the ligand binding site of platelet glycoprotein GPlb (p0p/B) abolishes this thrombus-promoting effect of rmSema7a. b) Representative IF images of the experiment in (a). c) rmSema7a does not affect adhesion or thrombus formation of GPlb-IL-4-tg platelets on collagen in flow at a shear rate of 1000 s−1. d) Representative IF images of the experiment from (c). Representative fluorescence images and mean surface coverage and relative thrombus volume are shown, as measured by integrated fluorescence intensity (IFI) per mm2±SEM (n 3/group)). e) GP-lb-IL4-tg animals were subjected to ischemia for 60 minutes and reperfusion for 120 minutes. They were injected with rmSema7a or rmIgG2A Fc control immediately before ischemia. f) Infarct sizes and correlate troponin I plasma levels were determined. g) Representative histological sections and h) Number of PNCs counted from myocardial tissue sections of GP-lb-IL4-tg injected with either rmSema7a or rmIgG2A Fc (control). i) Coimmunoprecipitation analysis between Sema7a and GPlb in blood and heart (AAR) was performed. Sema7a was affinity-precipitated using an anti-GPlb antibody, and GPlb was affinity-precipitated using a Sema7a antibody. Rat or mouse IgG was used as a negative control. Bound proteins were analyzed by immunoblotting. The same amount of protein was used in all input loads Of blood or heart lysates. j) Densitometric analysis was performed to quantify immunoblots. For comparisons in FIGS. 16 a and 16 c, the inventors performed one-way analyses of variance followed by Dunnett's tests on group WT +rmSema7a (data are means±SA; n=4/group). Comparisons in FIG. 16 f, h, i, and j were analyzed using Student's t tests (data are means±SA; n≥7/group; histology n=9/group; densitometric analysis n≥7/group). (*p<0.05, **p<0.01, and *** p<0.001 as indicated.

FIG. 17: Recombinant semaphorin 7A (rmSema7a) markedly enhances adhesion and thrombus formation of WT platelets on collagen in flow at a shear rate of 400 s⁻¹. Blockade of the ligand binding site of platelet GPlb (p0p/B) abolishes the thrombus-promoting effect of rmSema7a. Representative fluorescence images are shown, as well as mean surface coverage and relative thrombus volume measured by integrated fluorescence intensity (IFI) per mm². For comparisons in FIG. 17, the inventors performed one-way analyses of variance followed by Dunnett's tests on the WT+ rmSema7a group. (Data are means±SA; n≥3/group; *p<0.05, **p<0.01, and *** p<0.001 as indicated).

FIG. 18: Anti-Sema7a reduces myocardial RI injury, platelet activation, and PNC formation. Animals were injected with either anti-semaphorin 7A antibody (anti-Sema7a) or IgG control 5 minutes before the start of 120 minutes of reperfusion after 60 minutes of ischemia, and samples were collected after 1 minute or 120 minutes of reperfusion. (a) Representative TTC-stained myocardial infarction heart sections (blue/dark=retrograde Evans blue staining; red and white=AAR, white=infarct tissue) with (b) systematic evaluation of infarct sizes and correlating troponin I plasma levels. (c) Representative histological sections of WT animals injected with either IgG control or anti-Sema7a and (d) number of PNCs counted in mlardial tissue sections. (e) Representative flow cytometry blots of PNCs in blood from animals injected with sham, anti-Sema7a, or IgG control expressing GPlb (CD42b) and P-selectin (CD62P). Systematic evaluation of flow cytometric expression of mean fluorescence intensity (MFI) for f) GPlb (CD42b) and g) P-selectin (CD62P) and h) systematic evaluation of PNCs in % flow cytometrically in blood from animals injected with anti-Sema7a or IgG control at 1 min and 120 min. i) Systematic evaluation of flow cytometric expression of MFI for j) GPlb (CD42b) k) P-selectin (CD62P) and l) systematic evaluation of PNCs in % flow cytometrically in the AAR of animals injected with anti-Sema7a or IgG control after 1 min and 120 min. Comparisons in FIG. 18 b and d were analyzed by Student's t-tests (data are means±SA; n≥6/group; histology n=9/group). For FIG. 18 f, g, h, j, k, and l, the inventors used log-transformation of the data to fit normality. For log-transformed data, tests were performed on the log values, and the results are shown as geometric means and their 95% confidence intervals. (*p<0.05, **p<0.01, and *** p<0.001 as indicated).

FIG. 19: Anti-Sema7a attenuates the activation of integrin αIIbβ3 (JON/A). Animals were subjected to myocardial ischemia for 1 h followed by reperfusion for 2 h, and 5 min before the reperfusion phase, either anti-semaphorin 7A antibody (anti-Sema7a) or IgG control was injected. After 1 minute or 120 minutes of reperfusion, blood and myocardial tissue (AAR) sections were obtained. Representative flow cytometric PNC plots in blood and tissue from WT animals injected with either IgG control or anti-Sema7a showed integrin αIIbβ3 (JON/A) activation in PNCs after 1 minute and 120 minutes of reperfusion. Systematic evaluation of flow cytometric mean fluorescence intensities of activated αIIbβ3 (JON/A) on PNCs from mice injected with either anti-Sema7a or IgG control at 1 min and 120 min. For FIG. 19, the inventors used log-transformed data to fit normality. For log-transformed data, tests were performed on the log values, and the results are shown as geometric means and their 95% confidence intervals (*p<0.05 and ***p<0.001 as indicated, n≥5/group).

FIG. 20: Schematic representation of the role of Sema7a during myocardial ischemia-reperfusion. 1) During myocardial ischemia, shear stress and hypoxia lead to cleavage of Sema7a from the surface of erythrocytes (1a) as the major source of Sema7a within the vascular bed. 2) The released Sema7a then engages the glycoprotein lb receptor (2a) and activates platelets, which then exposes integrin receptors (2b), leading to platelet and blood platelet formation. 3) Activated platelets and PNCs migrate from the vasculature into the ischemic tissue, resulting in tissue damage and destruction.

### EXAMPLES

1. Material and Methods

Ethic Statement

Animal protocols were in accordance with the German guidelines for use of living animals and were approved by the Institutional Animal Care and the Regierungspräsidium Tübingen and Würzburg, and the Landesamt für Verbraucherschutz Niedersachsen. Approval for human sample processing was obtained by the ethics committee (Institutional Review Board) of the University of Tübingen. Samples of patients with myocardial infarction were obtained at presentation to the catheter laboratory and processed (Biobank: 266/2018BO1; Sema7a subanalysis: 266/2018BO2; Clinicaltrial.gov: NCT01417884). Patient samples before and after cardiac surgery were collected as part of the TüSep-Study (NCT02692118). Written informed consent was obtained from each patient before samples were taken.

Processing of Human Blood Samples

Human blood samples were taken during coronary intervention, at the end of cardiopulmonary bypass or during occlusion of the coronary arteries during off pump cardiac surgery and processed for flow cytometry. In addition, blood was centrifuged to obtain plasma samples, which were then stored and measured. Blood was also used for isolation of erythrocytes which were analyzed for SEMA7A expression or used in further experiments (Ethics approval 507/2017BO1)

Mice

Sema7a^{-/-}mice were generated, validated and characterized as described in the prior art. The corresponding WT controls were bred as littermates of the Sema7a mice. In a subset of experiments, a newly generated Sema7a flox mouse line (Sema7a^{loxP/loxP}/Ozgene) on a C57BL/6 background was crossed with the following Cre recombinase-positive mouse lines to obtain tissue-specific gene deletion: Erythrocyte-specific HbbCre+, myocardial cell-specific Myh6Cre+; endothelial cell-specific Tie2Cre+; and immune cell-specific LysMCre+. Tissue-specific gene deletions of Sema7a mouse lines (Sema7a^{loxP/loxP} HbbCre+; Sema7a^{loxP/loxP} Myh6Cre+, Sema7a^{loxP/loxP} Cre+, and Sema7a^{loxP/loxP} LysMCre+) were used in the experiments. Sema7a^{loxP/loxP} Cre-negative (−) littermates were used as controls. In a subset of experiments, the inventors used a functional GPlb knockout mouse line (GPlb-IL4tg) to test the interference of Sema7a with the GPlb receptor. In a subset of experiments, Sema7a^{loxP/loxP} HbbCre+ animals were reconstituted with recombinant mouse Sema7a (rmSema7a; R&D SYSTEMS, Minneapolis, USA) or recombinant mouse IgG2A Fc (rmlgG2A Fc; control).

Murine Myocardial Ischemia and Reperfusion Model

This animal model has been described in detail in the prior art. Subgroups of animals received either recombinant mouse Sema7a (rmSema7a) or recombinant mouse IgG2A Fc (rmlgG2A Fc; control) intravenously before the start of the experiment or Sema7a antibody (abcam ab23578, Cambridge, UK; anti-Sema7a) or as control rabbit IgG sc-2027 (Santa Cruz Biotechnology, Santa Cruz, USA) 5 minutes before the start of reperfusion.

Immunohistochemical Detection of Neutrophils, Platelets, and PNCs in Mouse Myocardial Tissue

The Vectastain ABC kit (Linaris, Wertheim, Germany) was used for immunohistochemical staining. After inhibition of the nonspecific binding sites with avidin blocking solution (Vector), the sections were incubated with primary antibodies (rabbit anti-mouse CD41, abcam, Cambridge, UK) overnight at 4° C. Tissue sections were then incubated with biotinylated rabbit anti-IgG for 1 hour, followed by Vectastain ABC reagent for 30 minutes, then developed over DAB substrate. For PMN staining, the procedure was repeated using rat anti-mouse neutrophil antibody (BioRad, Serotec, Puchheim, Germany) and HistoGreen as substrate (Linaris, Wertheim, Germany). Counterstaining was performed with Nuclear Fast Red (Linaris, Wertheim, Germany). Histological sections were analyzed for the presence of PNCs by manual counting within 3 independent tissue sections of each animal at a magnification of 400×400.

Troponin I Measurement

Troponin blood plasma levels of samples collected by central venous puncture after 120 minutes of reperfusion were measured for murine troponin I type 3 (TNNI3) using the SEA478Mu ELISA kit (Cloud-Clone Corp, Houston, USA).

Caspase 3 Staining and Caspase 3 ELISA

Human cardiac myocytes (HMC-c; PromoCell, Heidelberg, Germany) were grown to confluence on chamber slides followed by 6 hours of stimulation with rhSEMA7A, rhlgG1 Fc, BSA, or staurosporine (Sigma-Aldrich, Munich, Germany), 1 μg/ml each. After fixation, cells were stained with rabbit polyclonal anticaspase3 (abcam, Cambridge, UK).

Sema7a ELISA

Sema7a ELISAs were performed according to the manufacturer's instructions using the ELISA kit SEB448Hu for human and SEB448Mu for murine Sema7a (Cloud-Clone Corp., Houston, USA).

RT-qPCR

For RNA extraction, the inventors used the peqGOLD TriFast™ (Peqlab; Germany; Erlangen, Germany) according to the manufacturer's instructions. For cDNA synthesis, the Bio-Rad iScript kit (Bio-Rad; Germany; Munich) was used. Semiquantitative analysis of murine Sema7a was performed by real-time PCR using the sense primer 5′-GTG GGT ATG GGC TGC TTT TT-3′ (SEQ ID NO: 1) and the antisense primer 5′-CGT GTA TTC GCT TGG TGA CAT-3′ (SEQ ID NO: 2). The reference gene was the murine 18S rRNA gene with the following set of primers: sense 5′-GTA ACC CGT TGA ACC CCA TT-3′ (SEQ ID NO: 3) and antisense primer 5′-CCA TCC AAT CGG TAG CG-3′ (SEQ ID NO: 4).

Protein Analysis

Murine tissue was homogenized and resuspended in RIPA buffer. Samples were separated via SDS-polyacrylamide gels and blotted onto PVDF membranes. The following antibodies were used in murine samples: Anti-Sema7a antibody (ab23578; abcam, Cambridge, UK) and, to control loading conditions, GAPDH antibody (sc-25778; Santa Cruz Biotechnology, Santa Cruz, USA). For human samples, the inventors used a goat polyclonal anti-Sema7a antibody (AF2068; R&D SYSTEMS, Minneapolis, USA) and β-actin antibody (sc-130656; Santa Cruz Biotechnology). Bands were detected by chemiluminescence reaction of HRP-conjugated antibodies and developed with luminol reagent (sc-2048; Santa Cruz Biotechnology).

Coimmunoprecipitation and Immunoblotting

Coimmunoprecipitation (Co-IP) was performed according to the manufacturer's instructions using the Pierce Co-IP kit (Cat. No. 261498, Thermo Fisher Scientific, Waltham, USA). In brief, mouse samples were collected after 60 min of ischemia followed by 1 min (blood) or 15 min (cardiac tissue, AAR) of reperfusion. Then, 250 μl of citrate+blood was lysed in 1 ml of IP lysis/wash buffer and kept on ice. The AAR was incubated in 1 ml IP lysis/wash buffer and homogenized in a Precellys 24 (VWR/Peqlab, Erlangen, Germany) and kept at 4° C. for 60 min. All samples were centrifuged at 13,000×g for 10 minutes. Total protein of the lysates was measured using the Pierce™ BCA Protein Assay Kit (Thermo Scientific; cat. no. 23225) and analyzed in an Infinite® M200 Pro Plate Reader (Tecan, Männedorf, Switzerland). Ten micrograms of mouse monoclonal antibody against Sema7a (sc374432; Santa Cruz Biotechnology, Santa Cruz, USA) or 10 μg of rat monoclonal antibody against GPlb (p0p4) were immobilized on the Amino Link Plus coupling resin. As IgG control, 10 μg rat IgG (sc2016; Santa Cruz Biotechno-logy) or mouse IgG (X0943; Dako, Glostrup, Denmark) was used. For protein analysis, 30 μl per Co-IP eluate was applied to SDS-PAGE. For immunodetection, a rabbit polyclonal antibody against Sema7a (sc135263; Santa Cruz Biotechnology) and the described monoclonal antibody (p0p5) against GPlb were used. Species-matched alkaline phosphatase-conjugated secondary antibodies were used (goat anti-rabbit IgG-AP; sc-2007; Santa Cruz Biotechnology; and goat anti-rat IgG-AP; A18868; Thermo Fisher Scientific). Protein detection was performed with a BCIP/NBT substrate.

Cardiac Magnetic Resonance Imaging (MRI) and Assessment of RV and LV-EDV, -ESV, -EF, and mass by MRI

Animals underwent cardiac MRI at 22 weeks of age. Analysis was performed on a clinical workstation with semiautomated contour tracking software (CV142, release 4.1.8 (201), Circle Cardiovascular Imaging Inc., Calgary, Canada).

Flow Chamber Experiments

Platelet adhesion in flow was measured by perfusion of murine whole blood on collagen-coated coverslips (200 μg/ml fibrillar type I collagen) at 1000 s⁻¹ or 400 s⁻¹, as indicated. Platelets were labeled with a DyLight 488-conjugated anti-GPIX Ig derivative (0.2 ag/ml) and treated with rmSema7a or IgG2A Fc for 5 minutes at 37° C. before perfusion. In the case of treatment with GPlb-blocking antibody, 100 μg/mouse p0p/B Fab was injected intravenously 20 minutes before blood collection for the experiment. In further experiments, blood from animals with functional GPlb (GPlb-IL4tg) was used.

Flow Cytometric Analysis

For flow cytometric analysis, the following antibodies were used and freshly prepared before the experiment: rat anti-mouse Ly6G (Biolegend, clone 1A8) labeled with BV421 (Biolegend 127628), rat anti-mouse CD42b-FITC (Emfret, clone Xia.G5), rat anti-mouse CD62P (BD Pharmingen, clone RB40.34) labeled with Alexa Fluor 647 (BD 563674), rat anti-mouse activated GPIlb/IIIa-PE (Emfret, clone JON/A).

Gating Strategy

After staining, sampling focused peripheral granulocytes by their granularity and surface expression of the lymphocyte antigen-6 complex, locus G (Ly-6G), referred to as SSC/Ly-6G+. The presence of the platelet surface marker CD42b on the surface of SSC/Ly-6G+ events distinguished SSC/Ly-6G+/CD42b+ platelet-neutrophil complexes (PNCs) from freely circulating SSC/Ly-6G+/CD42b- PMNs. These two populations were tested for their representation of the surface transmembrane glycoprotein P-selectin (CD62P) and activated GPIlb/IIIa (clone JON/A) (not shown).

SEMA7A Cleavage from Erythrocytes

Erythrocytes were separated from human blood samples using MACS beads (MicroBeads Kit, Milenyi Biotec, Germany). Cells were quantified in a Neubauer chamber. 8×10⁸ erythrocytes were used per sample. Shear stress was induced by pulling and pushing the erythrocytes through a 27 gauge needle. For hypoxia exposure, cells were placed in hypoxic PBS medium exposed to hypoxia (8% O₂) in an Invivo2 400 hypoxia workstation (Ruskin Technology Ltd; Leeds). At the end of each experiment, samples were centrifuged at RT at 1200 rpm for 10 minutes, and SEMA7A concentration was measured in the supernatant using the SEB448Hu ELISA kit.

Intravital Microscopy

Mice were anesthetized, and cremasal tissue was dissected under a Nikon 20×water immersion lens (NA=0.32). Neutrophils were resuspended with 20 μl of anti-Ly6G (Biolegend 127608) and platelets with 20 μl of platelet-specific FITC-labeled X488 antibody (emfret, Eibelstadt, Germany) in 200 μl and administered i.v. Videos of postcapillary cremaster venules 20-40 μm in diameter were recorded using a Hamamatsu Orca Flash 4.0 camera mounted on a dual-emission image splitter (optoSplit II, Cairn Research; UK) at a rate of 16 frames/s (for videos totaling 10 seconds) and with a resolution of 2048×1024 pixels mounted on a Nikon Eclipse Ci-L microscope (Nikon, Dusseldorf; Germany) operated by NIS elements Ar software. Videos were recorded before administration of rmSema7a i.v., and videos were recorded at 20-second intervals for 5 minutes after incubation for 15 minutes. Neutrophil velocity (μm/sec) was calculated from manually tracked cells, stationary neutrophils were counted (cell count/mm²), platelet sedimentation on the vessel wall was measured (normalized MFI %/mm), transmigrated cells from the vasculature after 15 min exposure to rmSema7a were counted (cell count/mm²), the distance of transmigrated cells measured in μm in all videos was calculated using NIS elements Ar software 4.20-64 bit processed (Nikon, Dusseldorf, Germany).

Data Analysis

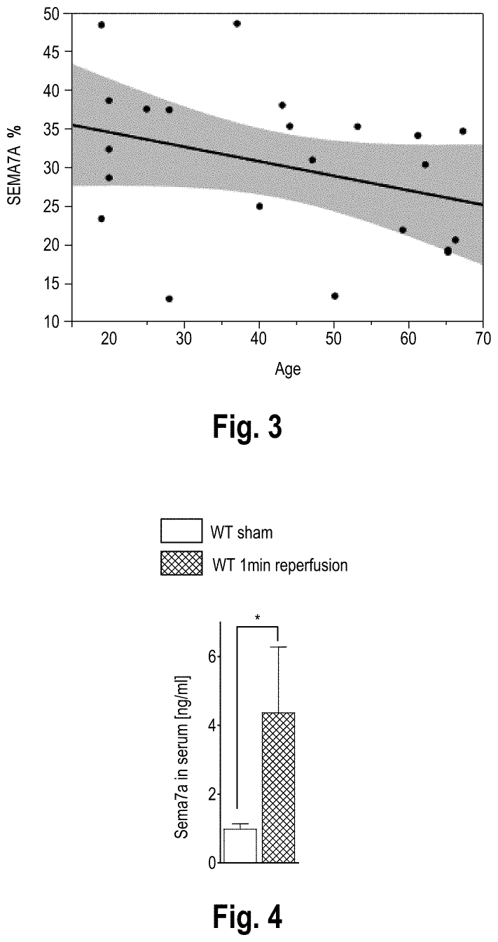

All data analysis was performed in collaboration with the Institute for Clinical Epidemiology and Applied Biometry at the University of Tübingen. Data are generally presented as bar graphs with means±SA. Normality of distribution was tested by skewness. Because testing for normality can easily lead to statistically nonsignificant but meaningless results for small sample sizes, as was the case in this study, the inventors also performed a visual inspection of the data distributions using histograms and attempted a log-transformation of the data to conform to normality when this inspection revealed skewed distributions. Tests of log values were performed for log-transformed data; the data are displayed on a logarithmic scale showing geometric means and their 95% confidence intervals. Overall, when comparing two groups, statistical tests were performed using Student's t-tests; for comparing multiple groups, the inventors performed one-way analyses of variance followed by Dunnett's tests. For FIG. 3: The SEMA7A % on human erythrocytes and age as input variables were analyzed by linear regression analysis and shown point-wise with 95% confidence bands, using healthy humans as donors and age as input variables. For all comparisons performed, p values are shown, and p values less than p<0.05 were considered statistically significant, indicated as p<0.05 (*); p<0.01 (**); and p<0.001 (***).

2. Results

Patients with myocardial ischemia show increased PNC formation and plasma SEMA7A

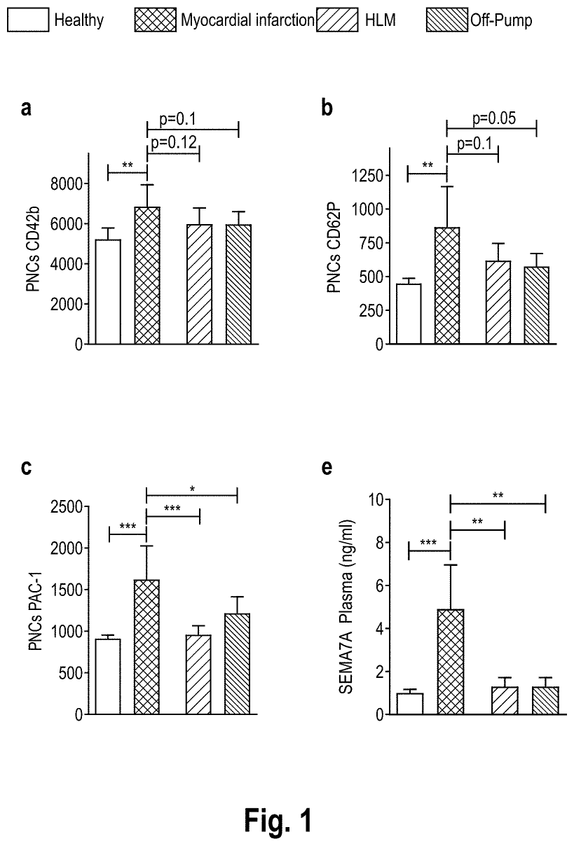

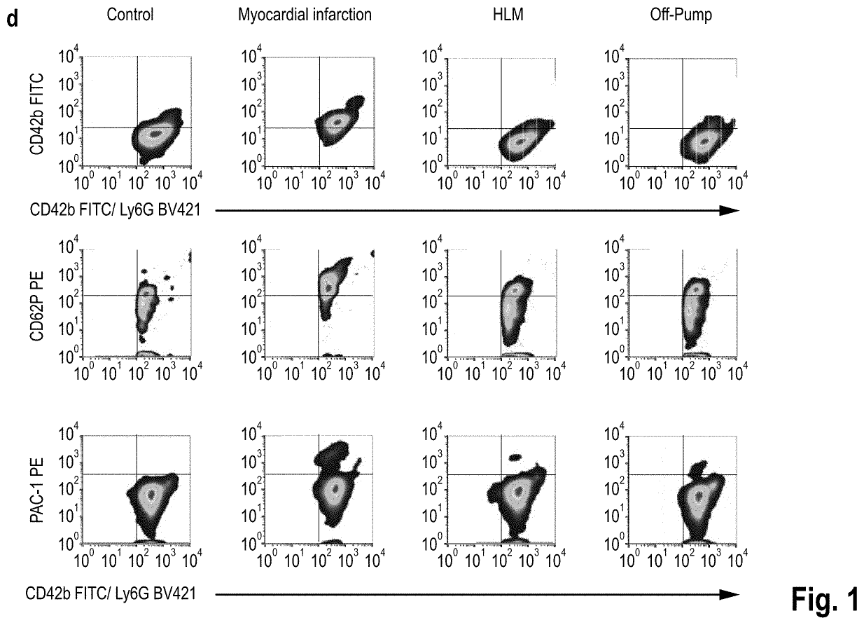

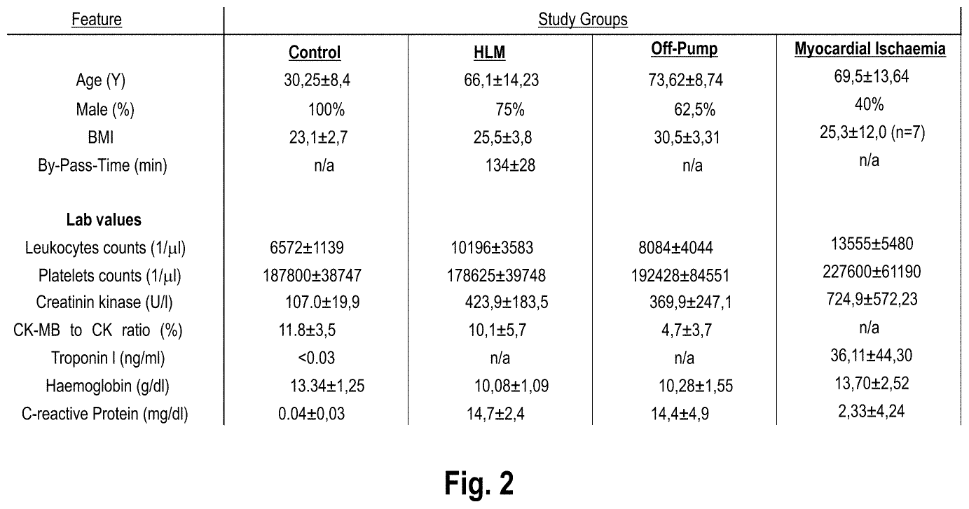

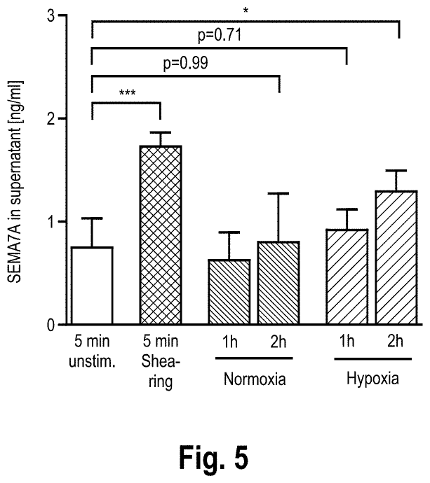

The number of PNCs increases in myocardial tissue during MIRI. The deleterious effects of PNCs have been demonstrated in other organs, such as the lung, where they increase inflammatory tissue damage, leading to impaired organ function. In an attempt to better understand the interaction of platelets and neutrophils and the formation of PNCs in inflammatory myocardial injury, the inventors collected blood samples from patients with active myocardial ischemia and examined them for the presence of PNCs by FACS analysis. The inventors compared these samples to patients undergoing cardiac surgery who were also undergoing reperfusion after extracorporeal circulation (HLM). These patients showed no evidence of active ischemia. In addition, the inventors obtained blood from cardiac surgery without reperfusion injury as well as healthy controls. The inventors found that the patients with acute MI had significantly more CD42b-positive neutrophils (i.e., PNCs) compared with healthy controls and patients undergoing cardiac surgery with or without extracorporeal circulation. Of note, platelets were fully activated in the conjugates, as shown by the marked activation of integrin IIb3 (PAC-1 binding) and CD62P exposure (FIG. 1 a-d). Given that reperfusion of ischemic tissue induces inflammation and the previous finding by the inventors that SEMA7A has significant pro-inflammatory capacity, the inventors also measured soluble SEMA7A in the blood of these patients. Indeed, SEMA7A increased in the plasma of MI patients but not in any of the other patient groups tested (FIG. 1 e, FIG. 2). The inventors also found that SEMA7A expression on erythrocytes was not age-dependent (FIG. 3). Experiments in mice revealed that plasma Sema7a increased very rapidly during cardiac ischemia, with a significant increase detected after only 1 minute of reperfusion (FIG. 4). SEMA7A was released from erythrocytes in response to shear stress or tissue hypoxia, both of which are present in affected areas during myocardial ischemia-reperfusion (FIG. 5).

Injection of Sema7a Worsens Myocardial IR Injury

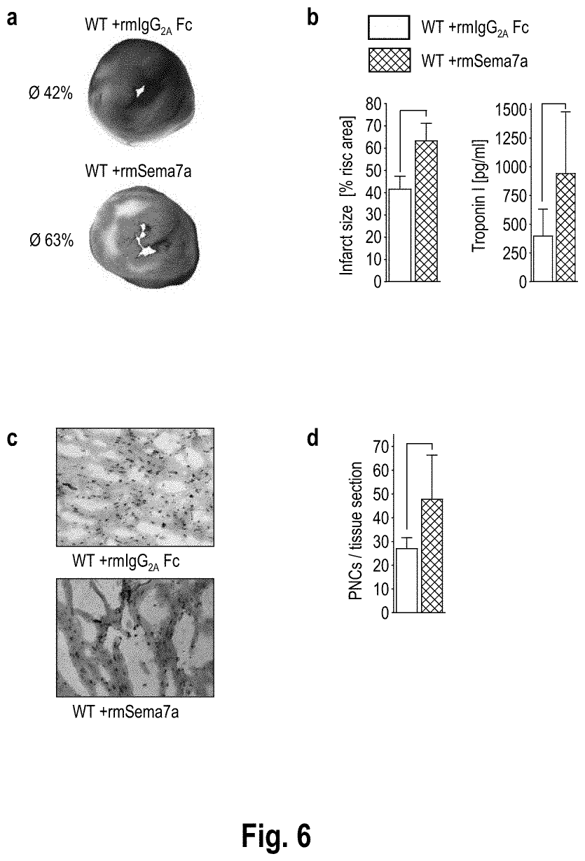

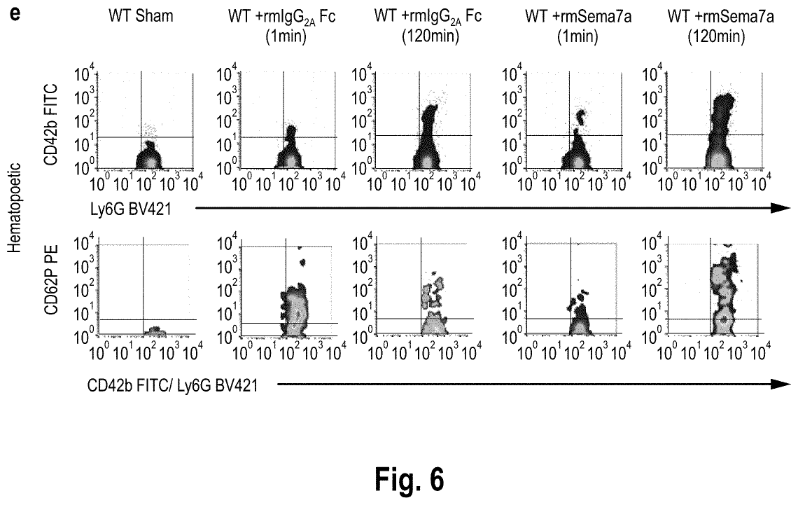

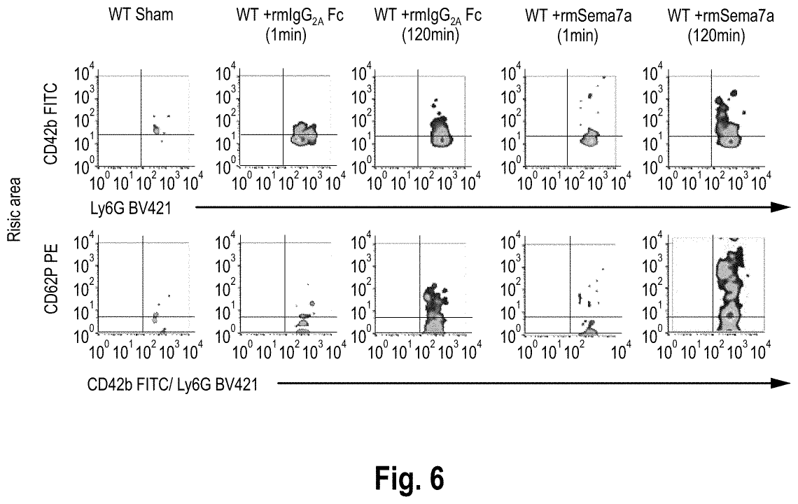

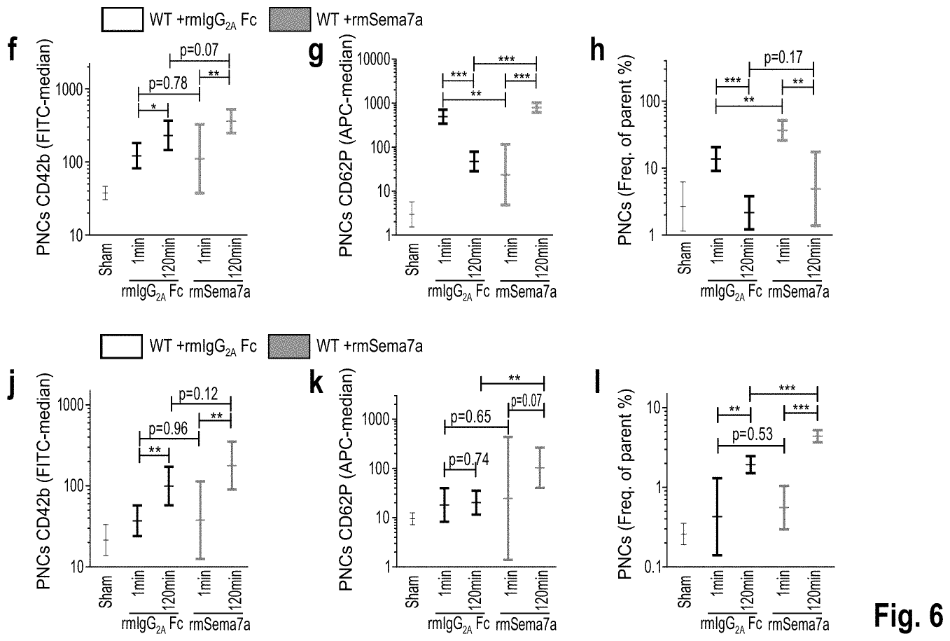

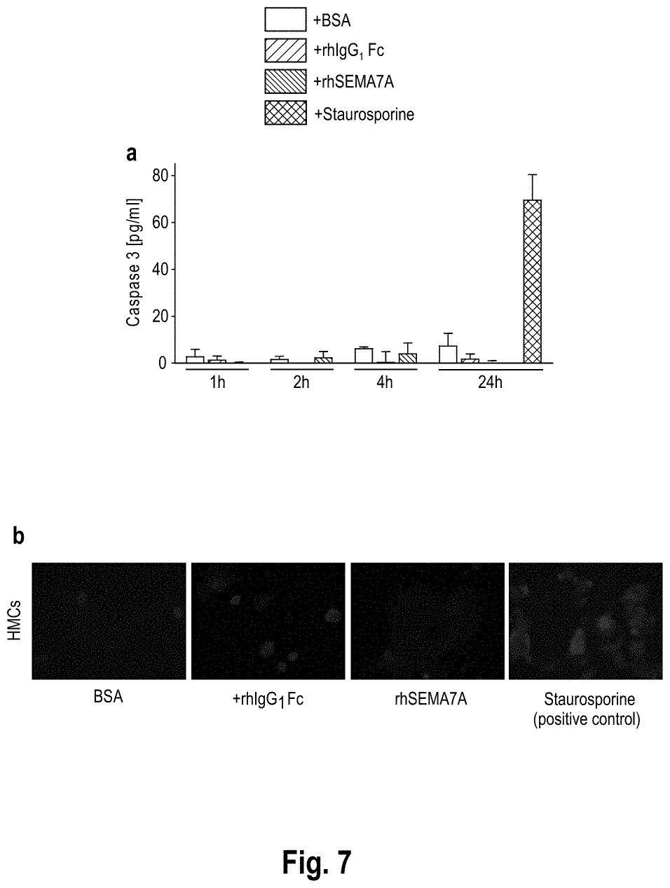

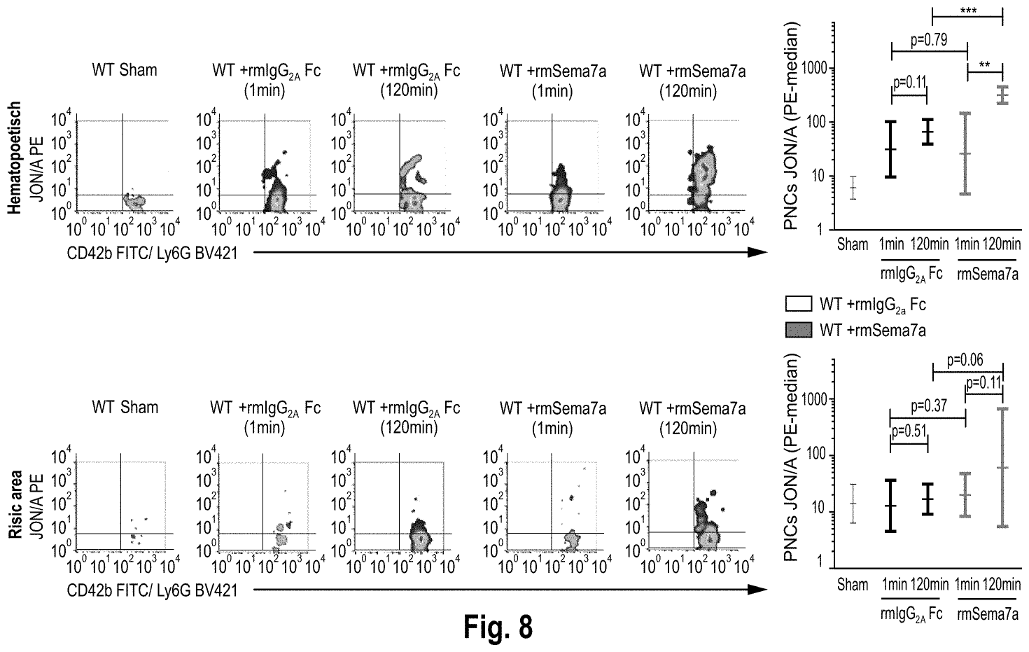

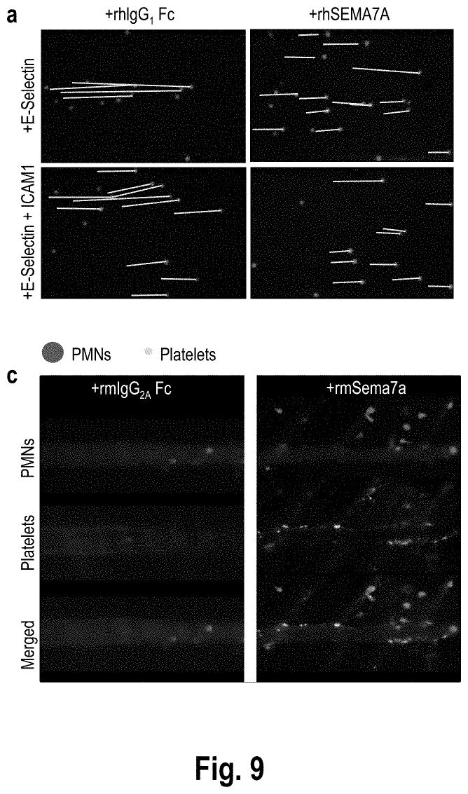

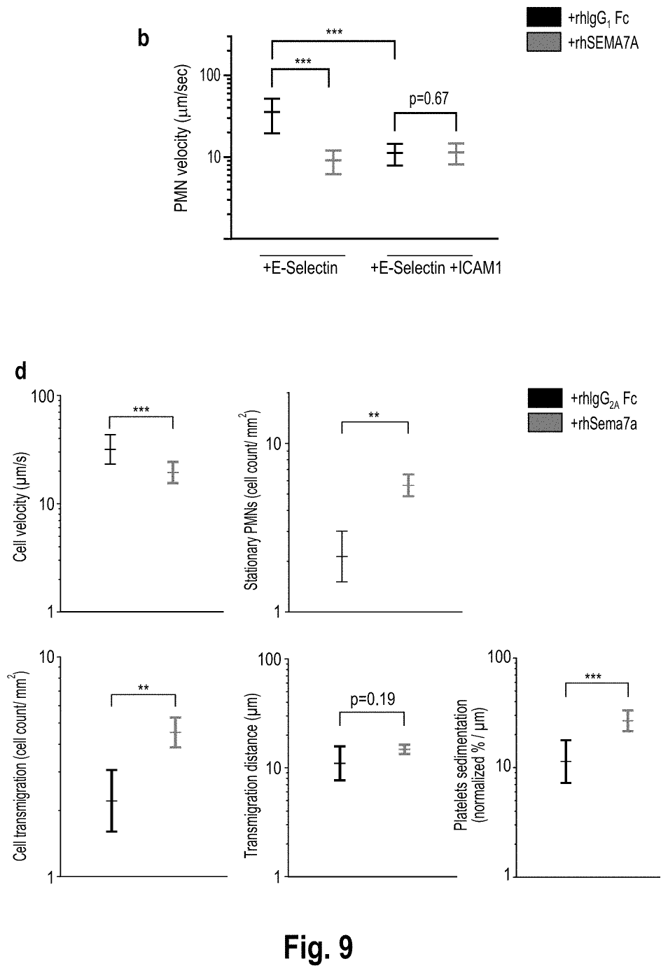

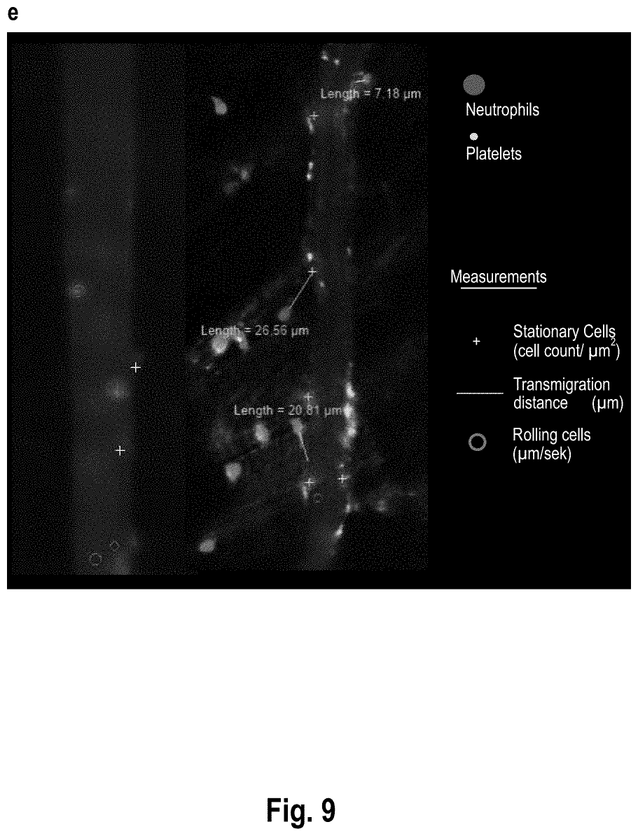

The large increase in Sema7a in plasma from patients and mice with myocardial ischemia raised the possibility that Sema7a plays a functional role in the progression of myocardial IR injury. To test this directly, the inventors injected recombinant Sema7a (rmSema7a fusion protein, 1 μg/mouse before reperfusion) into WT animals and found that this resulted in significantly increased infarct size compared with the corresponding IgG Fc control animals (rmlgG2A Fc) (FIG. 6 a,b). This finding correlated with increased troponin I, a marker of myocardial tissue damage. Histological sections showed increased tissue damage in Sema7a-injected animals compared with Fc controls and decreased numbers of PNCs in tissue areas at risk (FIG. 6 c,d). In cell culture experiments, the inventors found no direct pro-apoptotic effect of human Sema7a (rhSEMA7A) on cardiomyocytes as assessed by caspase 3 activation (FIG. 7 a,b). Having observed more PNCs in myocardial tissue and knowing the deleterious effects that these PNCs can have, the inventors next used flow cytometry to test for the presence of PNCs. The inventors found significantly increased numbers of PNCs in the blood of mice injected with rmSema7a, and the conjugates (FIG. 6 h) also showed stronger signals for the platelet markers GPlb, CD62P, and activated integrin αIIbβ3 (JON/A) (FIG. 6 e-g, FIG. 8). To test whether this was reflected by PNCs entering the area at risk (AAR), the inventors extracted this area and determined the number of PNCs in myocardial tissue. Using flow cytometry, the inventors found that more PNCs extravasated into the myocardium in the AAR in animals injected with rmSema7a and that conjugates showed significantly increased signals for GPlb, P-selectin, and activated integrin αIIbβ3 (JON/A) in neutrophils (FIG. 6 i-l, FIG. 8). The inventors also tested the effect of rmSema7a on neutrophil rolling and found that rmSema7a affected neutrophil adhesion in vitro and in vivo (FIG. 9). This is consistent with previous findings by the inventors that Sema7a expressed on the surface of endothelial cells increases transmigration of neutrophils into inflamed tissues.

Significantly Reduced Myocardial IR Injury in Sema7a^{-/-}Mice

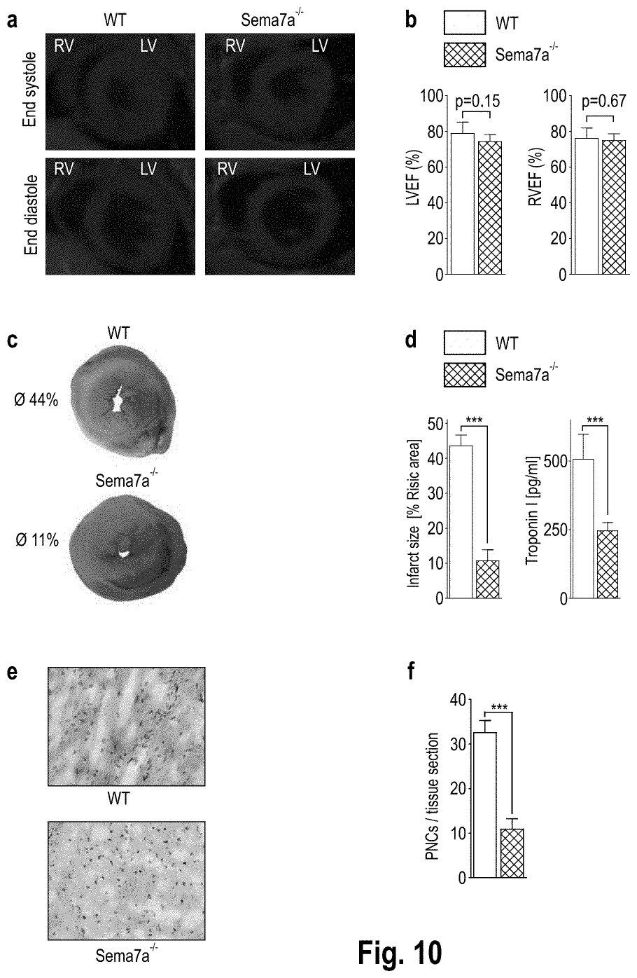

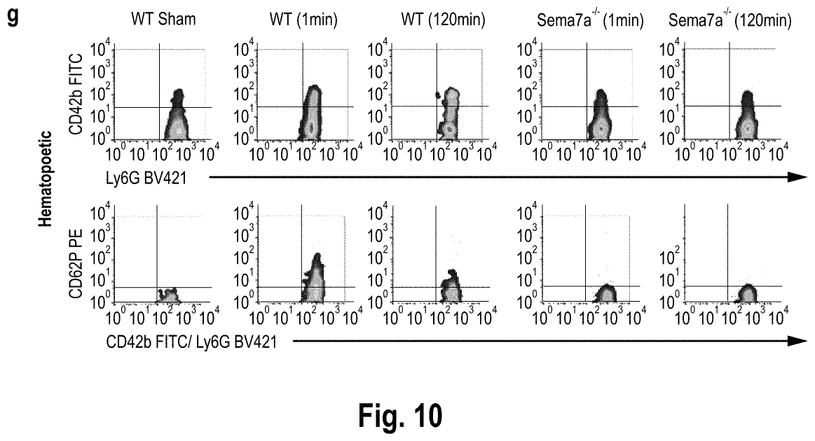

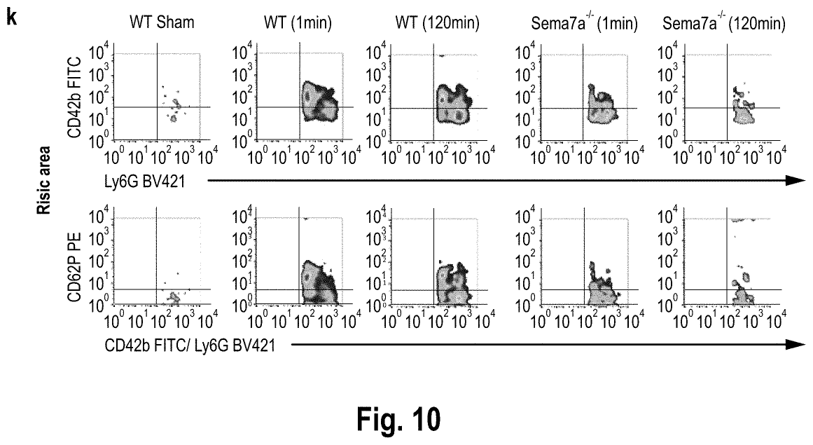

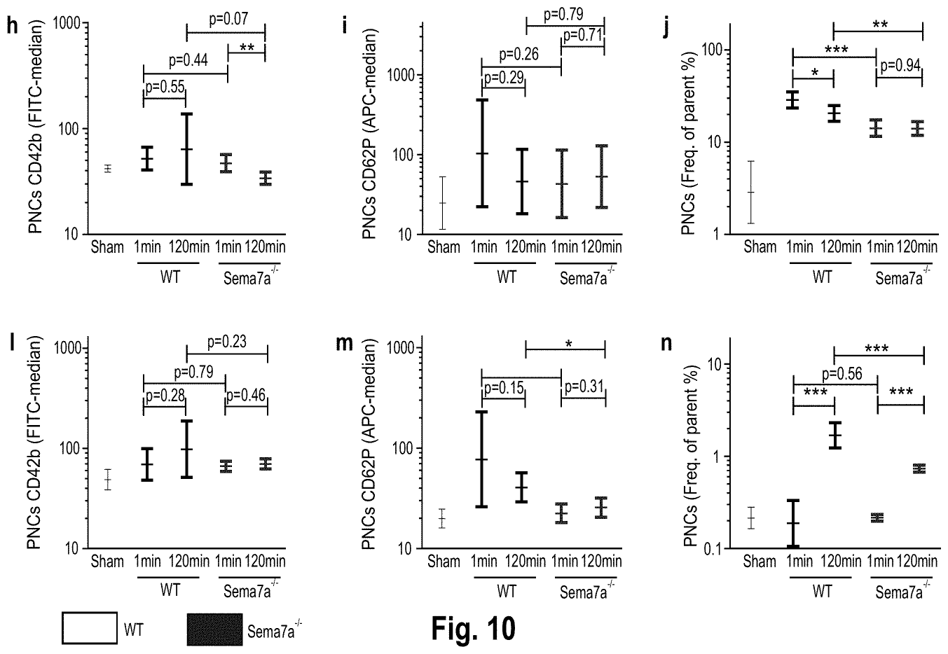

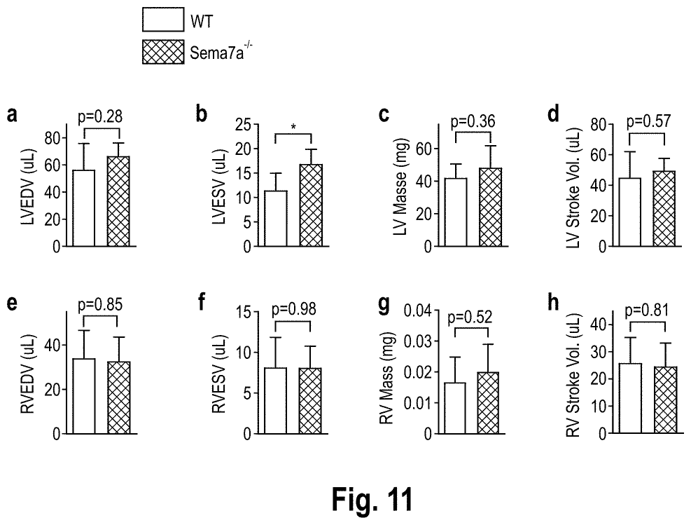

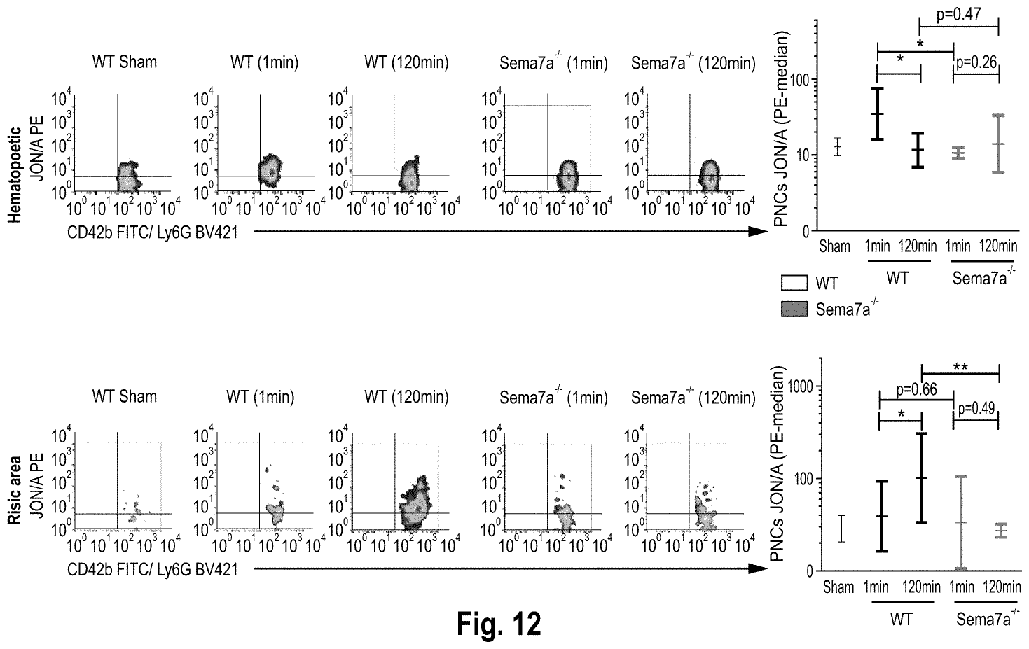

To further investigate the role of Sema7a in myocardial IR injury, the inventors used Sema7^{-/-}animals and their littermate controls. Because Sema7a is involved in fibrotic tissue transformation, the inventors first examined cardiac function in untreated WT and Sema7a^{-/-}animals using dynamic magnetic resonance imaging. The inventors found no differences in the anatomy or cardiac performance parameters of Sema7a^{-/-}mice compared with littermate controls (FIG. 10 a, b, FIG. 11). Next, the inventors exposed the Sema7a^{-/-}mice to MIR and found that they developed dramatically smaller infarcts and had decreased plasma troponin I compared with littermate controls (FIG. 10 c, d). Histological sections showed less tissue damage in Sema7a^{-/-}animals compared with littermate controls and a lower number of PNCs in vulnerable tissue areas (FIG. 10 e, f). Flow cytometric analysis showed lower numbers of PNCs (FIG. 10 j) in the blood of Sema7a^{-/-}animals compared with controls, and the conjugates showed lower levels of platelet activation markers (surface P-selectin and activated integrin αIIbβ3 (JON/A)) in the early phase after IR (FIG. 10 g-i, FIG. 12). The inventors again tested whether this was reflected within the PNCs in the AAR. The inventors extracted this area and counted the PNCs within the myocardial tissue. Using flow cytometry, the inventors found reduced numbers of PNCs within the AAR and conjugates showed reduced signals for GPlb, activated integrin αIIbβ3, and P-selectin on neutrophils in the AAR of Sema7a^{-/-}animals (FIG. 10 k-n, FIG. 12).

Red Blood Cell-Derived Semaphorin 7a is Central to Myocardial RI Injury

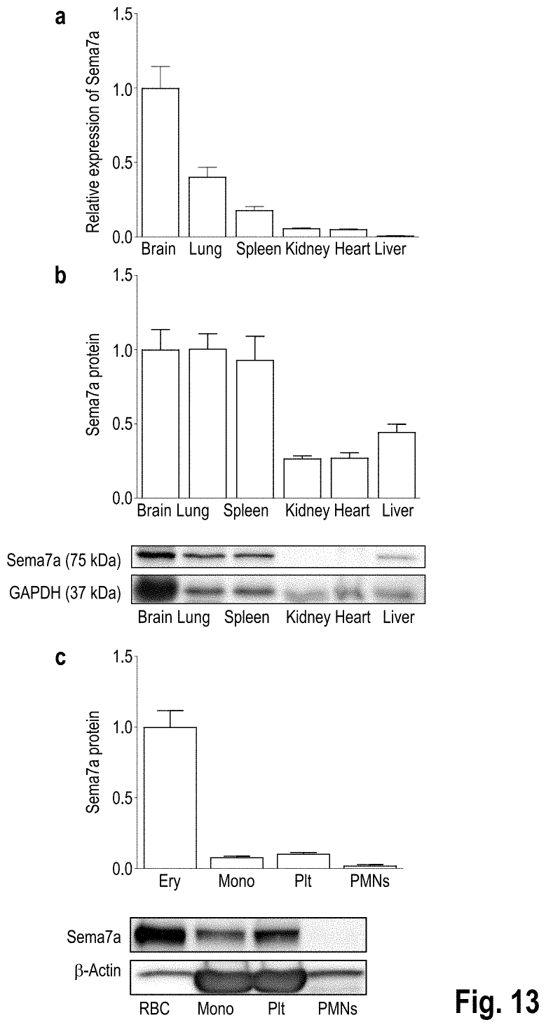

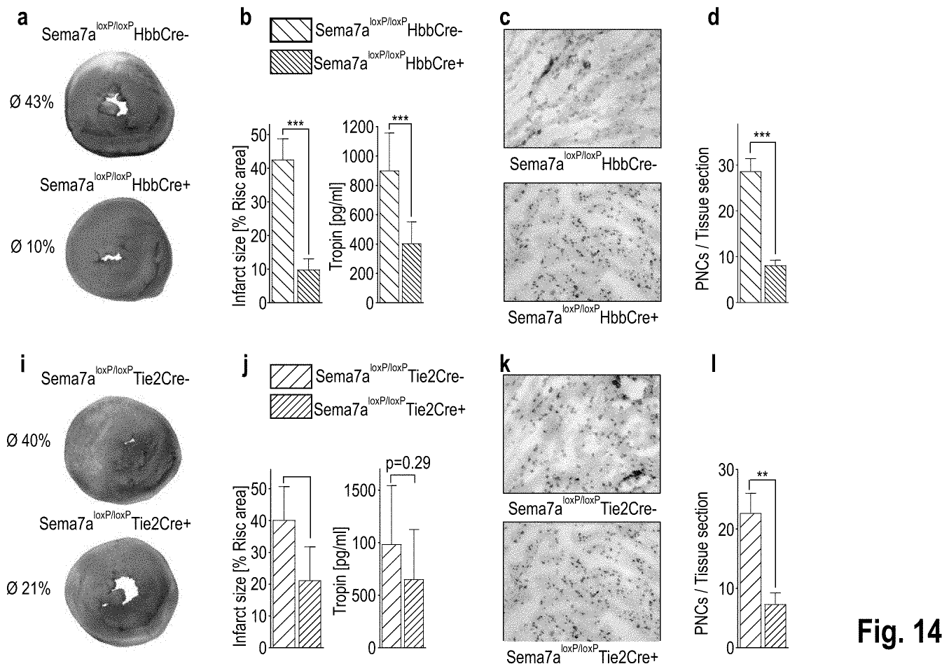

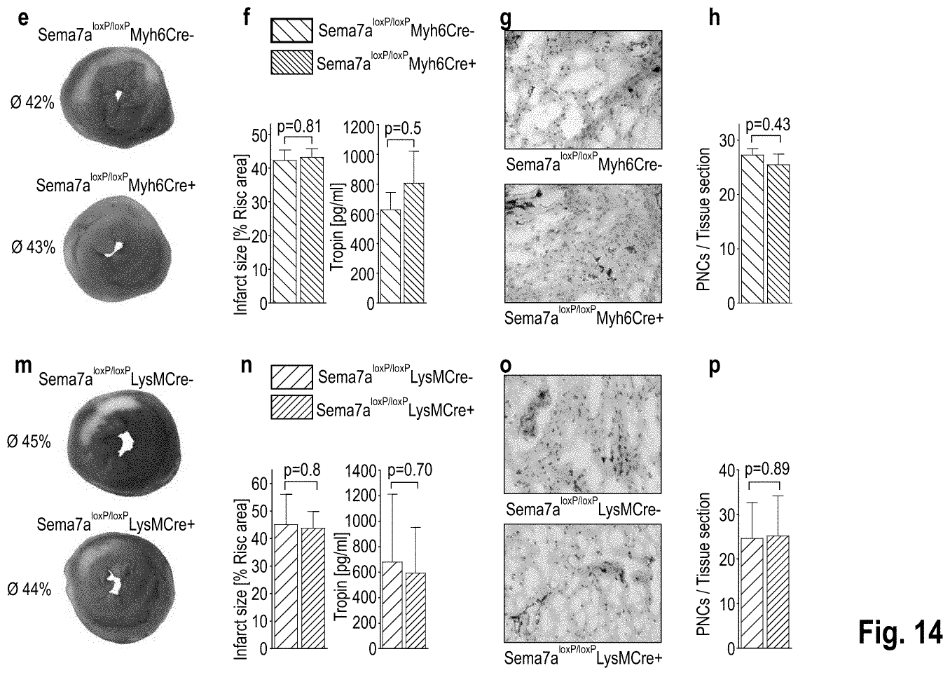

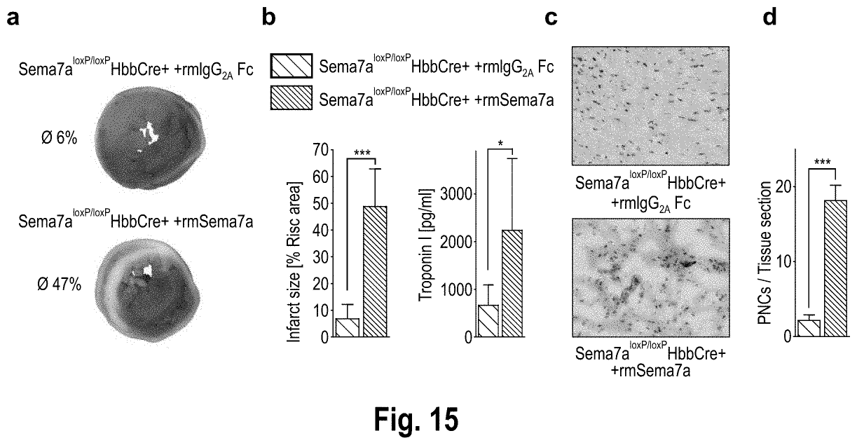

Next, the inventors sought to identify the cellular source of soluble Sema7a that mediates the observed pathogenic effect. Sema7a is expressed in various organs and tissues, and it is abundant on red blood cells (RBCs) and has low expression in myocardial tissue (FIG. 13). Therefore, the inventors generated animals with genetic deletion of Sema7a in endothelial cells (Sema7a^{loxP/loxP} Tie2Cre+), cardiomyocytes (Sema7a^{loxP/loxP} Myh6Cre+), erythrocytes (Sema7a^{loxP/loxP} HbbCre+), and immunocompetent cells (Sema7a^{loxP/loxP} LysMCre+) and fed them to the MIRI model. In the Sema7aloxP/loxPHbbCre+ animals, the inventors found a significant reduction in infarct size that correlated well with reduced plasma Tnl (FIG. 14 a,b). Immunohistochemical analysis showed reduced numbers of PNCs in the myocardial tissue of these animals compared with WT controls (FIG. 14 c,d). Injection of rmSema7a showed that these results were reversed in the Sema7aloxP/loxPHbbCre+ animals, although injection of Sema7a resulted in higher troponin levels in these animals (FIG. 15). In the Sema7a^{loxP/loxP} Myh6Cre+ animals, the inventors found no significant change in myocardial IR injury (FIG. 14 e,f) and no changes compared with littermate controls when PNCs in myocardial tissue were considered (FIG. 14 g,h). When Sema7a^{loxP/loxP} Tie2Cre+ animals were exposed to the same model, the inventors found significantly smaller infarcts compared with controls (FIG. 14 i,j). This protection was also reflected in a reduced number of PNCs, whereas the reduction in troponin I did not reach statistical significance (FIG. 14 k,l). In contrast, infarct size remained unchanged in Sema7a^{loxP/loxP} LysMCre-mice compared with controls (FIG. 14 m,n), as did troponin I concentration and the number of PNCs in myocardial tissue (FIG. 14 o,p). This approach demonstrated that expression of Sema7a on RBCs is critical for induction of PNC formation and myocardial tissue injury.

Sema7a Interacts with Platelet Glycoprotein Lb

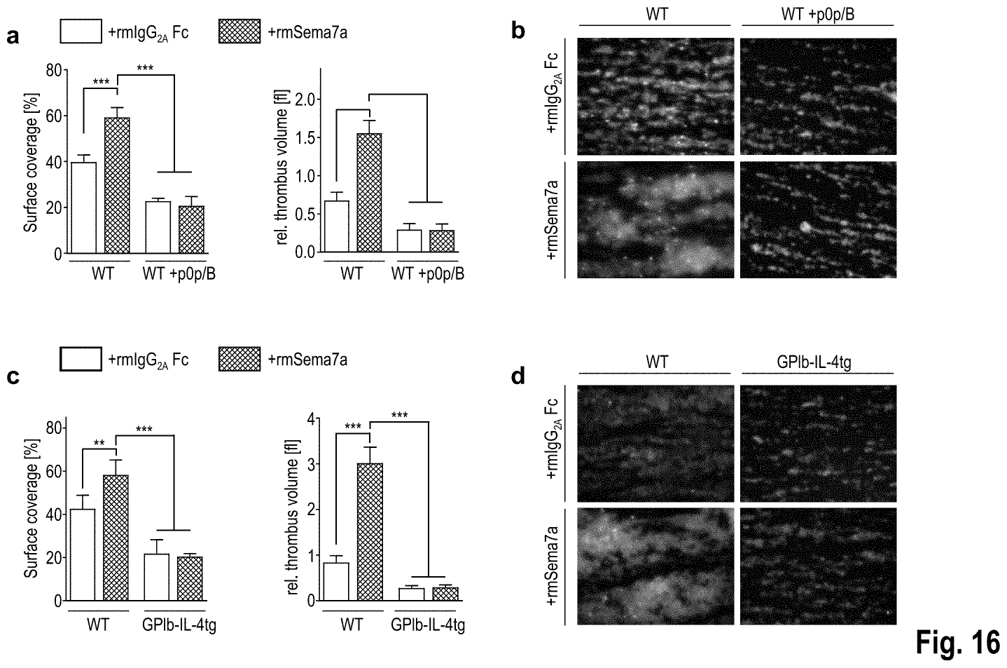

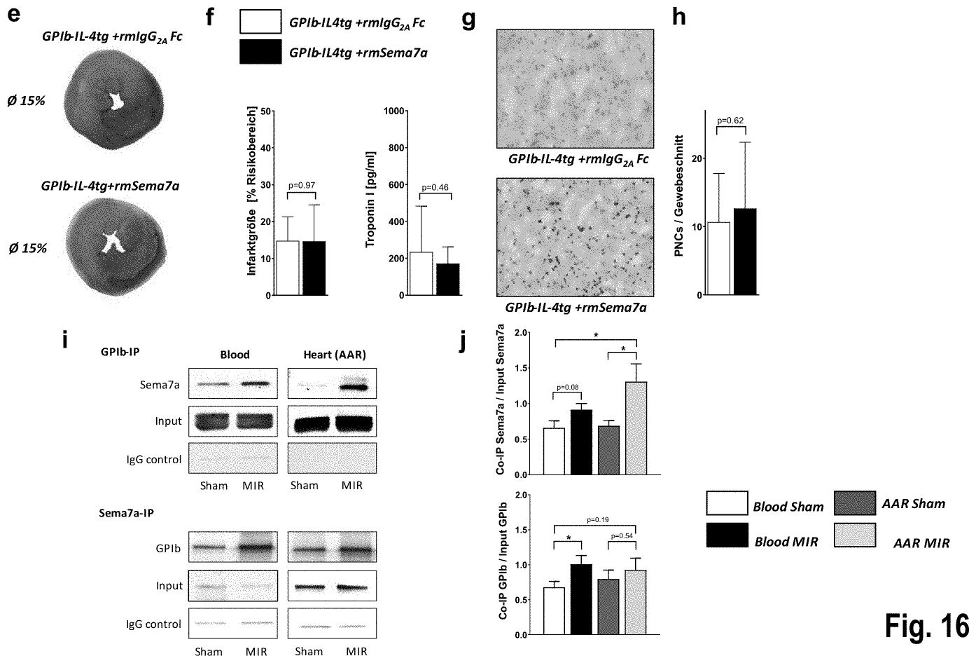

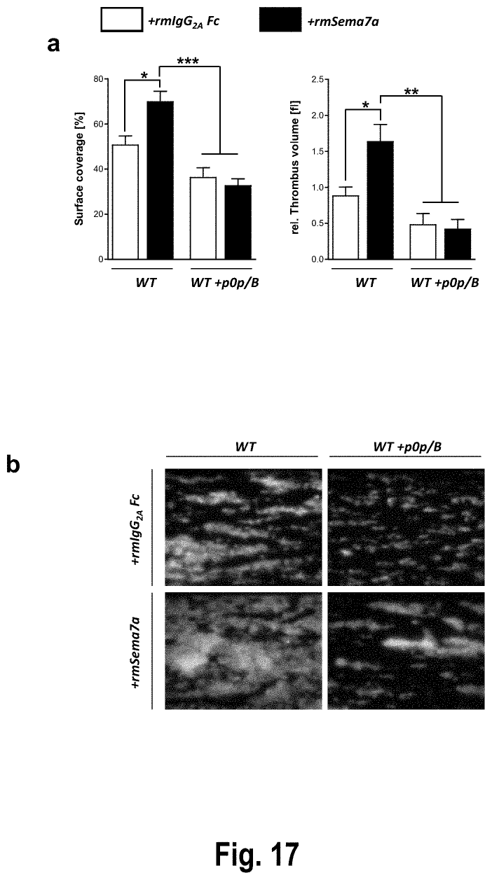

The above experiments have shown that Sema7a enhances platelet activation and PNC formation in the vicinity of myocardial IR. To test whether Sema7a acts directly on platelets, the inventors first examined its effects on platelet function in standard aggregometry. Unexpectedly, Sema7a did not induce any detectable platelet activation or aggregation at the 1 μg/ml concentration. This was also confirmed by flow cytometric analysis of platelet activation. Increasing concentrations of Sema7a had no effect on integrin-αIIbβ3 activation (JON/A-PE) or P-selectin exposure under static conditions. In stark contrast, profound prothrombotic activity of Sema7a was observed when thrombus formation was assessed on collagen in flow with a whole-blood perfusion system. At a medium to high shear rate (1000 s⁻¹), reflecting arterial blood flow, Sema7a significantly increased both platelet-covered surface area and thrombus volume (FIG. 16 a,b), and the same effect was observed at a low shear rate (400 s⁻¹, FIG. 17 a,b). The flow dependence of the prothrombotic Sema7a effect suggested a possible involvement of the vWF receptor GPlb-IX in this process, which is particularly important for thrombus formation under high shear conditions. To test this directly, the inventors inhibited GPlb-IX by adding Fab fragments of the antibody p0p/B, which completely blocks the ligand-binding site on the GPlb subunit of the receptor complex. Under these conditions, the thrombus-promoting effect of Sema7a was completely lost at both low and high shear rates, demonstrating that it was GPlb-dependent (FIG. 16 a,b). This result was also confirmed in mice in which the ectodomain of GPlbα was replaced by the human IL-4 receptor (GPlb-II-4tg) (FIG. 16 c,d), in which Sema7a did not induce an increase in surface coverage or thrombus volume in the whole blood perfusion system.

To test whether the thrombo-inflammatory effect of Sema7a myocardial RI injury also depends on platelet GPlb, the inventors first added GPlb-IL-4tg animals to the MIRI model. Strikingly, these animals exhibited significantly reduced infarct size compared with WT controls, which was also reflected in troponin I measurements. Moreover, treatment of these mutant animals with Sema7a did not increase MIRI, indicating that this pathogenic activity of Sema7a was completely GPlb-dependent (FIG. 16 e,f). In evaluating the tissue sections of the AAR of these animals, the inventors found a small number of PNCs within the myocardial tissue sections (FIG. 16 g,h). To test a possible interaction of Sema7a with GPlb, the inventors performed coimmunoprecipitation experiments. As a first step, the inventors immunoprecipitated Sema7a from blood and cardiac muscle tissue samples and blotted for the presence of GPlb. Indeed, GPlb co-immunoprecipitated with Sema7a in blood and particularly myocardial AAR in response to MIR. The inventors then reversed this approach with immunoprecipitation of GPlb and blotting for Sema7a. Again, the inventors found an interaction of Sema7a with GPlb in blood and the AAR in response to MIR (FIG. 16 i,j).

Anti-Sema7a Treatment Reduces PNC Formation and Myocardial IR Injury

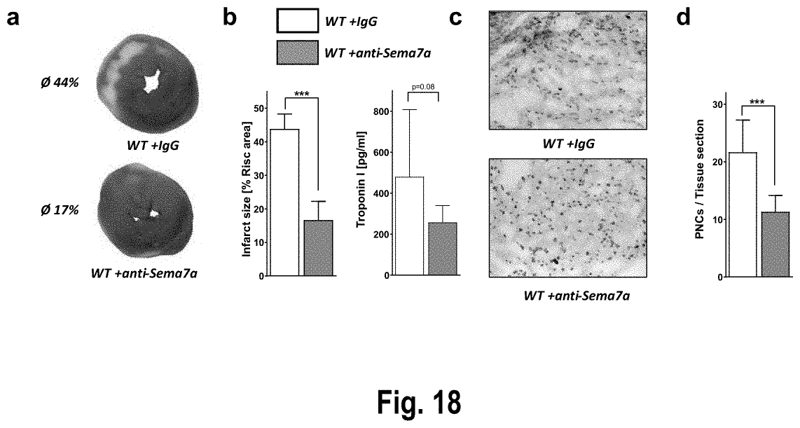

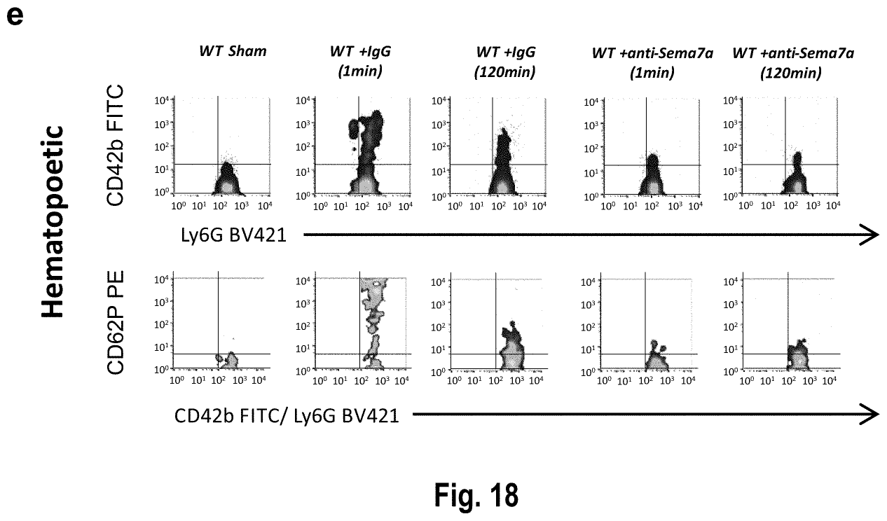

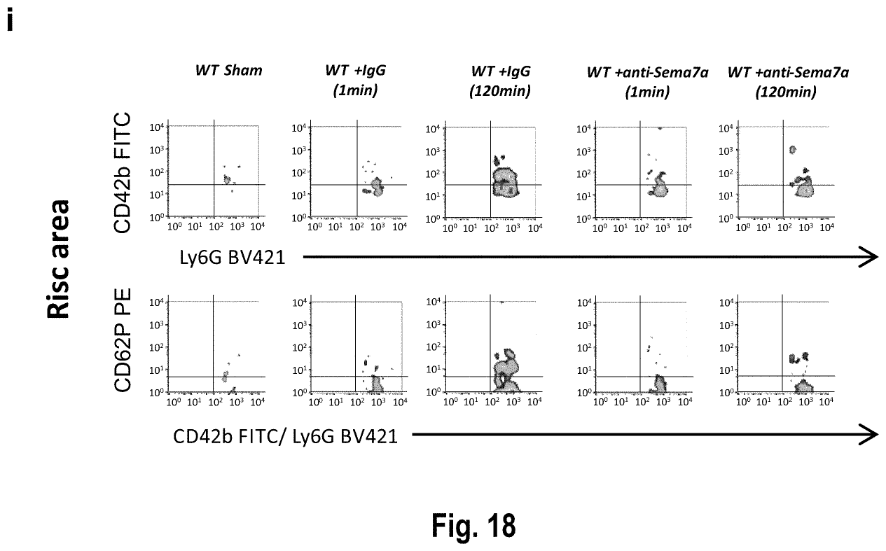

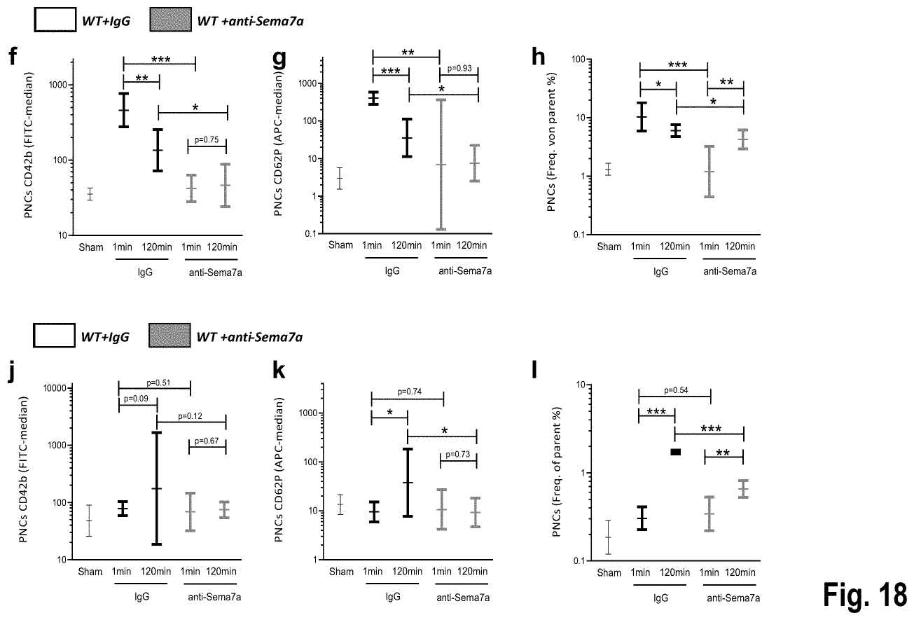

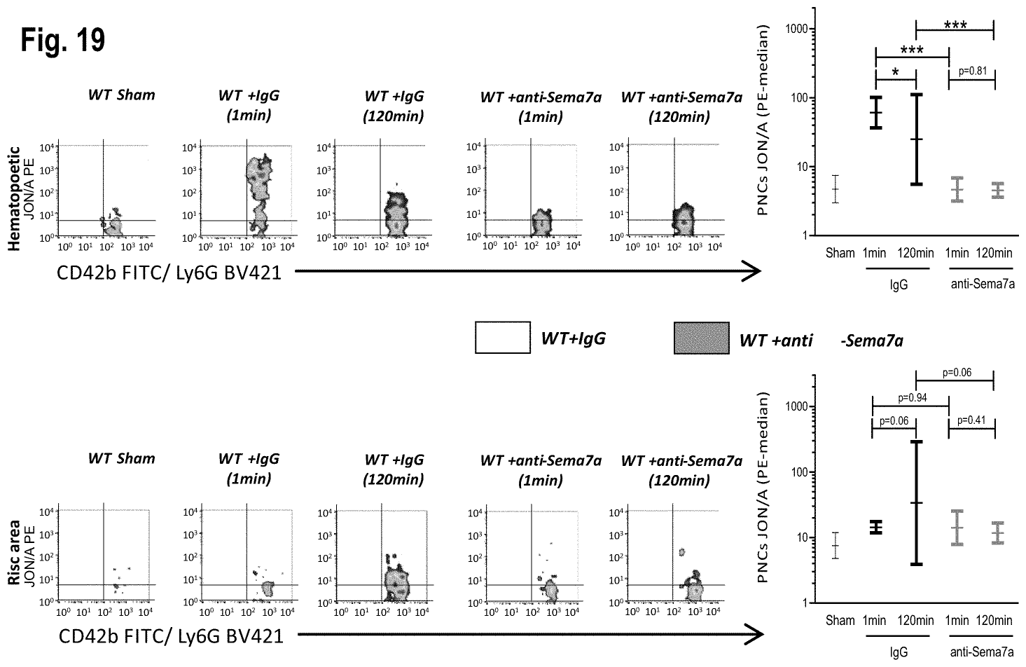

To test whether inhibition of endogenous Sema7a affects MIRI, the inventors injected a function-blocking anti-Sema7a antibody or IgG control (1 μg/mouse) before starting reperfusion. Indeed, anti-Sema7a treatment resulted in a reduction in infarct size and a reduction in troponin I compared with animals injected with an IgG control (FIG. 18 a,b). AAR histological sections showed less tissue damage in anti-Sema7a-injected animals compared with IgG control animals, with a lower number of PNCs in vulnerable tissue areas (FIG. 18 c,d). Flow cytometry showed increased numbers of CD42b-positive and P-selectin-positive neutrophils in the blood of the IgG-injected animals at baseline after IR, which was not observed in the anti-Sema7a-injected animals (FIG. 18 e-g, FIG. 19). In addition, the inventors found a significantly reduced number of PNCs in the blood of these anti-Sema7a-injected animals (FIG. 18 h). Analysis of the AAR revealed a significant increase in PNCs with fully activated platelets (indicated by high signals for GPlb, activated integrin αIIbβ3, and P-selectin) at the later time point (120 min) in the IgG-control animals that was virtually absent in the anti-Sema7a-injected animals (FIG. 18 j-k, FIG. 19), and a lower number of PNCs within the AAR (FIG. 18 l). These data demonstrate that PNC formation has proximate effects on myocardial tissue damage in experimental myocardial IR and can be effectively prevented by inhibition of endogenous Sema7a.

3. Discussion

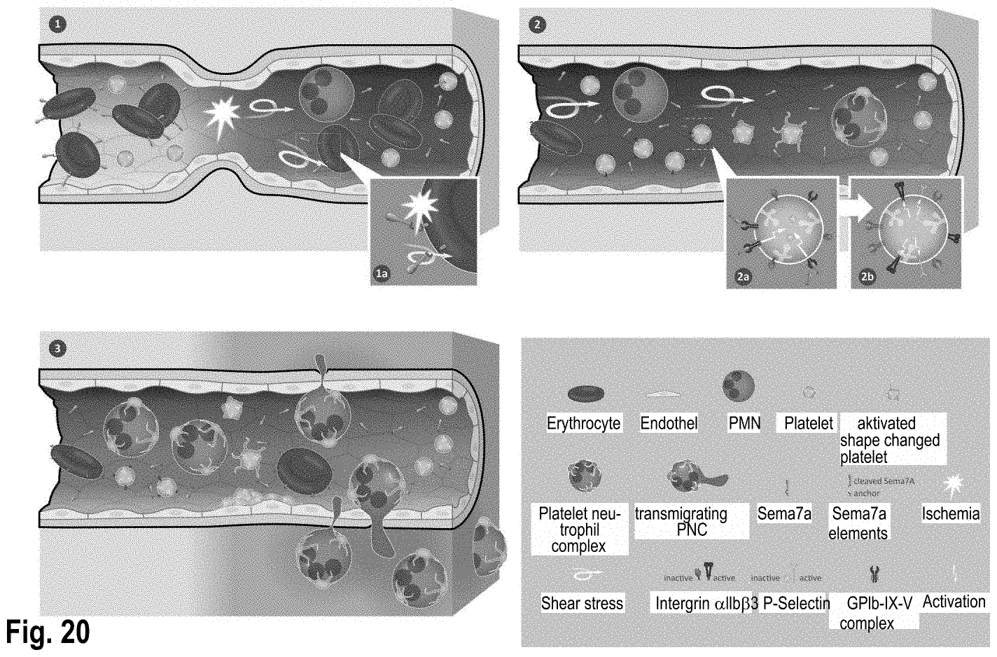

Myocardial ischemia followed by reperfusion remains one of the most significant health problems worldwide. Intervention to recanalize the occluded coronary artery is a critical part of the initial therapy for this condition and greatly improves the overall patient outcome. After occlusion, subsequent reperfusion injury to the myocardium is the result of an inflammatory response that affects a large proportion of patients with MI and can then lead to severe myocardial dysfunction. The inventors demonstrated that the neuronal guidance protein semaphorin 7a released from the red blood cell membrane is a mediator of inflammatory myocardial injury. The inventors further provide evidence that Sema7a interacts with platelet GPlb to promote platelet-neutrophil complex formation and translocation into the affected myocardium, thereby enhancing myocardial injury. To illustrate the role of Sema7a on platelets and the effect of Sema7a on MIRI, the inventors provided a sketch in FIG. 20. The results indicate that a strategy to affect the Sema7a-GPlb interaction may lead to reduced myocardial injury and improved myocardial outcome after MI and should therefore be pursued as a therapeutic strategy in the future.

4. Conclusion

Based on their knowledge of the molecular basis of postischemic tissue damage, particularly in the form of reperfusion injury, the inventors were able for the first time to develop an agent that enables targeted prophylaxis and therapy.

What is claimed is:

1. A method for the treatment and/or prophylaxis of postischemic tissue injury comprising administering a therapeutically effective amount of an inhibitor of SEMA7A into a mammal in need thereof.

2. The method according to claim 1, wherein the post-ischemic tissue damage is a reperfusion damage.

3. The method according to claim 2, wherein the reperfusion damage is myocardial reperfusion damage.

4. The method according to claim 1, wherein said inhibitor is configured to modify the interaction of SEMA7A with the platelet surface.

5. The method according to claim 1, wherein said inhibitor is configured to modify the interaction of SEMA7A with GPlb.

6. The method according to claim 1, wherein the inhibitor is selected from the group consisting of: antibody, antibody fragment, soluble SEMA7A receptor, antisense nucleic acid, siRNA, small molecule compound, and combinations thereof.

7. The method according to claim 6, wherein the antibody is a function-inhibiting antibody.

8. The method of claim 1, wherein the mammal is a human.

9. The method according to claim 8, wherein the administration of the inhibitor is into a myocardial infarction patient or a patient at risk of myocardial infarction.

10. The method according to claim 1, wherein the administration of the inhibitor modifies the interaction of SEMA7A with the platelet surface.

11. The method according to claim 1, wherein the administration of the inhibitor modifies the interaction of SEMA7A with GPlb.

12. A pharmaceutical composition for the treatment or prophylaxis of post-ischemic tissue damage comprising an inhibitor of semaphorin 7a (SEMA7A) and a pharmaceutically acceptable carrier.

13. A method for the manufacture of a medicament for the treatment and/or prophylaxis of post-ischemic tissue injury, comprising formulating an inhibitor of SEMA7A into a pharmaceutically acceptable carrier.
